(19) United States
(12) Patent Application Publication
(10) Pub. No.: US 2026/0354899 A1
(43) Pub. Date: Nov. 12, 2026

(54) BALLOON CATHETER FOR USE WITHIN AN INTRAVASCULAR LITHOTRIPSY DEVICE

(71) Applicant: Boston Scientific Scimed Inc., Maple Grove, MN (US)

(72) Inventors: Eric Schultheis, San Clemente, CA (US); Alvin Salinas, San Marcos, CA (US)

(73) Assignee: Boston Scientific Scimed Inc., Maple Grove, MN (US)

(21) Appl. No.: 19/664,678

(22) Filed: Apr. 30, 2026

Related U.S. Application Data
(60) Provisional application No. 63/801,520, filed on May 7, 2025.

(51) Int. Cl. A61B 18/24; A61B 18/26; A61M 25/10; A61B 18/00

(52) U.S. Cl. CPC A61B 18/245; A61B 18/26; A61M 25/10; A61B 2018/00184; A61B 2018/00404; A61B 2018/263; A61M 2025/1065; A61M 2025/1084; A61M 2025/1093; A61M 2210/12

(57) ABSTRACT

A catheter system for treating a treatment site includes an energy source, a catheter shaft, a guidewire lumen, a balloon, and a first energy guide. The energy source generates energy. The guidewire lumen is positioned at least partially within the catheter shaft, is substantially rigid and has a distal end. The balloon includes a balloon proximal end that is coupled to the catheter shaft, a balloon distal end that is secured to the guidewire lumen, and a balloon wall that defines a balloon interior. The balloon is selectively inflatable with a catheter fluid to move to an inflated state wherein the balloon wall is positionable substantially adjacent to the treatment site. The balloon is selectively movable between an inverted configuration, and an extended configuration. The first energy guide selectively receives the energy from the energy source, and guides the energy from the energy source into the balloon interior.

### CROSS-REFERENCE TO RELATED APPLICATIONS

This application claims the benefit of priority under 35 U.S.C. §119 of U.S. Provisional Application No. 63/801,520, filed May 7, 2025, the entire disclosure of which is hereby incorporated by reference.

### BACKGROUND

Vascular lesions within blood vessels and/or heart valves in the body can be associated with an increased risk for major adverse events, such as myocardial infarction, embolism, deep vein thrombosis, stroke, and the like. Severe vascular lesions, such as severely calcified vascular lesions, can be difficult to treat and achieve patency for a physician in a clinical setting. One type of severe vascular lesion that can be particularly difficult to treat is commonly referred to as a chronic total occlusion (or “CTO”), which encompasses a complete blockage in one of the blood vessels of a patient that restricts blood flow to and/or from the heart of the patient.

Vascular lesions may be treated using interventions such as drug therapy, balloon angioplasty, atherectomy, stent placement, and vascular graft bypass, to name a few. Such interventions may not always be ideal or may require subsequent treatment to effectively address the lesion.

Intravascular lithotripsy is a treatment system and methodology that has taken on increased importance in recent years for treating vascular lesions within a body of a patient. During an intravascular lithotripsy procedure, a balloon catheter can be employed to treat the vascular lesions at a treatment site within the body of the patient. However, presently available intravascular lithotripsy treatment systems and methodologies typically are not specifically configured to effectively address chronic total occlusions.

Therefore, it is desired to provide a catheter system that is specifically configured to treat chronic total occlusions, as well as other types of vascular lesions, as part of an intravascular lithotripsy treatment system and methodology.

The present invention is directed toward a catheter system for treating a treatment site within or adjacent to a vessel wall of a blood vessel, or within or adjacent to a heart valve, within a body of a patient. In various embodiments, the catheter system includes an energy source, a catheter shaft, a guidewire lumen, a balloon, and a first energy guide. The energy source generates energy. The guidewire lumen is positioned at least partially within the catheter shaft. The guidewire lumen is substantially rigid and has a distal end. The balloon includes a balloon proximal end that is coupled to the catheter shaft, a balloon distal end that is secured to the guidewire lumen, and a balloon wall that defines a balloon interior. The balloon is configured to retain a catheter fluid within the balloon interior. The balloon is selectively inflatable with the catheter fluid to move to an inflated state wherein the balloon wall is positionable substantially adjacent to the treatment site. The balloon is selectively movable between an inverted configuration in which at least a portion of the balloon extends farther distally than the distal end of the guidewire lumen, and an extended configuration in which no portion of the balloon is positioned more distally than where the balloon distal end is secured to the guidewire lumen. The first energy guide selectively receives the energy from the energy source, and guides the energy from the energy source into the balloon interior.

In many embodiments, selective, mechanical movement of the guidewire lumen relative to the catheter shaft selectively moves the balloon between the inverted configuration and the extended configuration.

In some embodiments, selective, mechanical movement of the guidewire lumen relative to the catheter shaft in a distal direction selectively moves the balloon from the inverted configuration to the extended configuration.

In certain embodiments, selective, mechanical movement of the guidewire lumen relative to the catheter shaft in a proximal direction selectively moves the balloon from the extended configuration to the inverted configuration.

In some embodiments, when the balloon is in the inverted configuration, the balloon distal end is bullnose-shaped.

In several embodiments, the balloon distal end has a rounded, forward perimeter, and a flat portion that extends radially inwardly from the forward perimeter, the forward perimeter having a radius of curvature that is between approximately 0.01 millimeters and one millimeter.

In certain embodiments, the radius of curvature of the forward perimeter is less than approximately 0.8 millimeters.

In other embodiments, the radius of curvature of the forward perimeter is less than approximately 0.5 millimeters.

In still other embodiments, the radius of curvature of the forward perimeter is less than approximately 0.2 millimeters.

In yet other embodiments, the radius of curvature of the forward perimeter is less than approximately 0.1 millimeters.

In many embodiments, the flat portion extends radially inwardly from the forward perimeter between approximately 0.1 millimeters and five millimeters.

In some embodiments, the flat portion extends radially inwardly from the forward perimeter at least approximately 0.2 millimeters.

In other embodiments, the flat portion extends radially inwardly from the forward perimeter at least approximately 0.4 millimeters.

In still other embodiments, the flat portion extends radially inwardly from the forward perimeter at least approximately one millimeter.

In yet other embodiments, the flat portion extends radially inwardly from the forward perimeter at least approximately two millimeters.

In still yet other embodiments, the flat portion extends radially inwardly from the forward perimeter at least approximately three millimeters.

In certain embodiments, the balloon has a balloon diameter, and a ratio of the balloon diameter to the flat portion is less than approximately 8:1.

In some embodiments, the ratio of the balloon diameter to the flat portion is less than approximately 5:1.

In other embodiments, the ratio of the balloon diameter to the flat portion is less than approximately 3:1.

In many embodiments, when the balloon is in the inverted configuration, the balloon further includes an inverted region that extends proximally from the flat portion.

In certain embodiments, when the balloon is in the inverted configuration, the balloon further includes a fold back region that extends distally from the inverted region.

In some embodiments, the balloon has a balloon inner surface, and the fold back region is configured such that the balloon inner surface is secured to the guidewire lumen.

In certain embodiments, the balloon inner surface is secured to the guidewire lumen with a securing bond.

In several embodiments, when the balloon is in the extended configuration, the balloon further includes an extended region that extends distally from the flat portion.

In some embodiments, the catheter shaft has a shaft diameter, and the balloon has a balloon diameter that is approximately equal to the shaft diameter.

In certain embodiments, the balloon is formed from a non-resilient material such that the balloon diameter remains constant when the balloon moves to the inflated state.

In some embodiments, the balloon has no tapered portions along a balloon length of the balloon from the balloon proximal end to the balloon distal end.

In several embodiments, the catheter system further includes a first emitter including a guide distal end of the first energy guide, and a corresponding first plasma target.

In some embodiments, the first energy guide emits the energy from the energy source from the guide distal end and toward the first plasma target so that plasma is generated in the catheter fluid within the balloon interior.

In certain embodiments, the plasma generated in the balloon interior subsequently generates pressure waves that are directed toward the balloon wall.

In some embodiments, the first emitter is at a first emitter station that is located at a first longitudinal position relative to a balloon length of the balloon.

In certain embodiments, the first emitter is a forward-firing emitter that directs energy from the plasma toward the balloon distal end.

In some embodiments, the forward-firing emitter is configured to direct the energy from the plasma in a direction substantially parallel to a longitudinal axis of the catheter shaft.

In other embodiments, the first emitter is a lateral-firing emitter that directs energy from the plasma toward the balloon wall.

In certain embodiments, the lateral-firing emitter is configured to direct the energy from the plasma in a direction that is at an angle relative to a longitudinal axis of the catheter shaft.

In many embodiments, the catheter system further includes a second energy guide that selectively receives the energy from the energy source, the second energy guide guiding the energy from the energy source into the balloon interior.

In several embodiments, the catheter system further includes a second emitter including a guide distal end of the second energy guide, and a corresponding second plasma target.

In some embodiments, the second energy guide emits the energy from the energy source from the guide distal end and toward the second plasma target so that plasma is generated in the catheter fluid within the balloon interior.

In certain embodiments, the plasma generated in the balloon interior subsequently generates pressure waves that are directed toward the balloon wall.

In some embodiments, the second emitter is at a second emitter station that is located at a second longitudinal position relative to the balloon length of the balloon, the second longitudinal position being different than the first longitudinal position.

In certain embodiments, the second emitter is a forward-firing emitter that directs energy from the plasma toward the balloon distal end.

In some embodiments, the forward-firing emitter is configured to direct the energy from the plasma in a direction substantially parallel to a longitudinal axis of the catheter shaft.

In other embodiments, the second emitter is a lateral-firing emitter that directs energy from the plasma toward the balloon wall.

In certain embodiments, the lateral-firing emitter is configured to direct the energy from the plasma in a direction that is at an angle relative to a longitudinal axis of the catheter shaft.

In many embodiments, the balloon distal end has a rounded, forward perimeter, and a flat portion that extends radially inwardly from the forward perimeter, the forward perimeter and the flat portion of the balloon being encompassed within a balloon distal region.

In some embodiments, the first emitter is positioned proximally relative to the balloon distal region.

In several embodiments, the catheter system further includes a flushing system that selectively removes contamination from the balloon interior by enabling the catheter fluid to selectively flow into and out of the balloon interior.

In some embodiments, the flushing system includes an inflation lumen positioned at least partially within the catheter shaft that directs catheter fluid into the balloon interior.

In certain embodiments, the flushing system further includes an inflation conduit that is in fluid communication with the inflation lumen, and a fluid outflow conduit that is in fluid communication with the catheter fluid in the balloon interior.

In some embodiments, the flushing system further includes a valve that selectively regulates flow of the catheter fluid through the fluid outflow conduit.

In one embodiment, the valve is a one-way valve.

In various embodiments, the energy source is a light source, and the first energy guide includes an optical fiber.

The present invention is also directed toward a catheter system for treating a treatment site, the catheter system including an energy source that generates energy; a catheter shaft; a guidewire lumen that is positioned at least partially within the catheter shaft, the guidewire lumen being substantially rigid and having a distal end; a balloon including a balloon proximal end, a balloon distal end that is secured to the guidewire lumen, and a balloon wall that defines a balloon interior, the balloon being configured to retain a catheter fluid within the balloon interior, the balloon being selectively inflatable with the catheter fluid to move to an inflated state wherein the balloon wall is positionable substantially adjacent to the treatment site, the balloon being selectively movable between an inverted configuration in which at least a portion of the balloon extends farther distally than the distal end of the guidewire lumen, and an extended configuration in which no portion of the balloon is positioned more distally than where the balloon distal end is secured to the guidewire lumen; and a first energy guide that selectively receives the energy from the energy source, the first energy guide guiding the energy from the energy source into the balloon interior; wherein selective, mechanical movement of the guidewire lumen relative to the catheter shaft selectively moves the balloon between the inverted configuration and the extended configuration.

The present invention is further directed toward a method for treating a treatment site including steps of generating energy with an energy source; positioning a guidewire lumen at least partially within a catheter shaft, the guidewire lumen being substantially rigid and having a distal end; coupling a balloon proximal end of a balloon to the catheter shaft; securing a balloon distal end of the balloon to the guidewire lumen; selectively inflating the balloon with a catheter fluid to move to an inflated state, the balloon including a balloon wall that defines a balloon interior, the balloon being configured to retain the catheter fluid in the balloon interior; positioning the balloon substantially adjacent to the treatment site; selectively moving the balloon between an inverted configuration in which at least a portion of the balloon extends farther distally than the distal end of the guidewire lumen, and an extended configuration in which no portion of the balloon is positioned more distally than where the balloon distal end is secured to the guidewire lumen; selectively receiving the energy from the energy source with a first energy guide; and guiding the energy from the energy source into the balloon interior with the first energy guide.

The present invention is also directed toward a method for treating a treatment site including steps of generating energy with an energy source; positioning a guidewire lumen at least partially within a catheter shaft, the guidewire lumen being substantially rigid and having a distal end; securing a balloon distal end of a balloon to the guidewire lumen; selectively inflating the balloon with a catheter fluid to move to an inflated state, the balloon including a balloon wall that defines a balloon interior, the balloon being configured to retain the catheter fluid in the balloon interior; positioning the balloon substantially adjacent to the treatment site; selectively, mechanically moving the guidewire lumen relative to the catheter shaft so as to selectively move the balloon between an inverted configuration in which at least a portion of the balloon extends farther distally than the distal end of the guidewire lumen, and an extended configuration in which no portion of the balloon is positioned more distally than where the balloon distal end is secured to the guidewire lumen; selectively receiving the energy from the energy source with a first energy guide; and guiding the energy from the energy source into the balloon interior with the first energy guide.

In some examples, a catheter may include a catheter shaft, a guidewire lumen that is positioned at least partially within the catheter shaft, the guidewire lumen being substantially rigid and having a distal end, a balloon including a balloon proximal end that is coupled to the catheter shaft, a balloon distal end that is secured to the guidewire lumen, and a balloon wall that defines a balloon interior, the balloon being configured to retain a catheter fluid within the balloon interior, the balloon being selectively expandable with the catheter fluid to move to an expanded state wherein the balloon wall is positionable substantially adjacent to a treatment site, the balloon being selectively movable between an inverted configuration in which at least a portion of the balloon extends farther distally than the distal end of the guidewire lumen, and an extended configuration in which no portion of the balloon is positioned more distally than where the balloon distal end is secured to the guidewire lumen, and a first energy guide that is configured to receive energy from an energy source, the first energy guide guiding the energy from the energy source into the balloon interior.

In addition to or as an alternative to any of the examples herein, mechanical movement of the guidewire lumen relative to the catheter shaft may move the balloon between the inverted configuration and the extended configuration.

In addition to or as an alternative to any of the examples herein, mechanical movement of the guidewire lumen relative to the catheter shaft in a distal direction may move the balloon from the inverted configuration to the extended configuration.

In addition to or as an alternative to any of the examples herein, mechanical movement of the guidewire lumen relative to the catheter shaft in a proximal direction may move the balloon from the extended configuration to the inverted configuration.

In addition to or as an alternative to any of the examples herein, when the balloon is in the inverted configuration, the balloon distal end may be bullnose-shaped.

In addition to or as an alternative to any of the examples herein, when the balloon is in the inverted configuration, the balloon may further include an inverted region that extends proximally from a flat portion.

In addition to or as an alternative to any of the examples herein, when the balloon is in the inverted configuration, the balloon may further include a fold back region that extends distally from the inverted region.

In addition to or as an alternative to any of the examples herein, the balloon may have a balloon inner surface and the fold back region is configured such that the balloon inner surface is secured to the guidewire lumen.

In addition to or as an alternative to any of the examples herein, when the balloon is in the extended configuration, the balloon may further include an extended region that extends distally from a flat portion.

In addition to or as an alternative to any of the examples herein, the catheter shaft may have a shaft diameter, and the balloon has a balloon diameter that is approximately equal to the shaft diameter.

In addition to or as an alternative to any of the examples herein, the balloon may be formed from a non-resilient material such that a balloon diameter remains constant when the balloon moves to the expanded state.

In addition to or as an alternative to any of the examples herein, the balloon may have no tapered portions along a balloon length of the balloon from the balloon proximal end to the balloon distal end.

In addition to or as an alternative to any of the examples herein, the catheter may include a first emitter configured to emit the energy from the first energy guide so that a wave is generated in the catheter fluid within the balloon interior, wherein the first emitter may be a forward-firing emitter that directs energy from the wave toward the balloon distal end.

In some examples, a catheter system may include an energy source that generates energy and the catheter of any one of configurations herein.

In some examples, a method of adjusting the balloon of the catheter of any of the examples herein may include moving the balloon between the inverted configuration in which at least a portion of the balloon extends farther distally than the distal end of the guidewire lumen and an extended configuration in which no portion of the balloon is positioned more distally than where the balloon distal end is secured to the guidewire lumen.

In some examples, a catheter system may include an energy source that generates energy, a catheter shaft, a guidewire lumen that is positioned at least partially within the catheter shaft, the guidewire lumen being substantially rigid and having a distal end, a balloon including a balloon proximal end that is coupled to the catheter shaft, a balloon distal end that is secured to the guidewire lumen, and a balloon wall that defines a balloon interior, the balloon being configured to retain a catheter fluid within the balloon interior, the balloon being selectively expandable with the catheter fluid to move to an expanded state wherein the balloon wall is positionable substantially adjacent to a treatment site, the balloon being selectively movable between an inverted configuration in which at least a portion of the balloon extends farther distally than the distal end of the guidewire lumen, and an extended configuration in which no portion of the balloon is positioned more distally than where the balloon distal end is secured to the guidewire lumen, and a first energy guide that is configured to receive the energy from the energy source, the first energy guide guiding the energy from the energy source into the balloon interior.

In addition to or as an alternative to any of the examples herein, mechanical movement of the guidewire lumen relative to the catheter shaft may move the balloon between the inverted configuration and the extended configuration.

In addition to or as an alternative to any of the examples herein, when the balloon is in the inverted configuration, the balloon distal end may be bullnose-shaped.

In addition to or as an alternative to any of the examples herein, when the balloon is in the inverted configuration, the balloon may further include an inverted region that extends proximally from a flat portion.

In addition to or as an alternative to any of the examples herein, the catheter shaft may have a shaft diameter, and the balloon may have a balloon diameter that is approximately equal to the shaft diameter.

In addition to or as an alternative to any of the examples herein, the catheter system may further include a first emitter configured to emit the energy from the first energy guide so that a wave is generated in the catheter fluid within the balloon interior, wherein the first emitter may be a forward-firing emitter that directs energy from the wave toward the balloon distal end.

In some examples, a method for treating a treatment site within or adjacent to a vessel wall of a blood vessel, or within or adjacent to a heart valve, within a body of a patient may include positioning a balloon of a catheter substantially adjacent to the treatment site, moving the balloon between an inverted configuration in which at least a portion of the balloon extends farther distally than a distal end of a guidewire lumen of the catheter and an extended configuration in which no portion of the balloon is positioned more distally than where a balloon distal end is secured to the guidewire lumen, and providing energy from an energy source to an energy guide with a distal end positioned in an interior of the balloon to create a wave within the balloon.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a simplified schematic cross-sectional view illustration of an embodiment of a catheter system in accordance with various embodiments herein, the catheter system including a catheter that includes an inflatable balloon having features of the present invention, which is configured to treat a treatment site within a body of a patient, the balloon being shown in an inverted configuration;

FIG. 2A is another simplified schematic cross-sectional view illustration of the catheter system illustrated in FIG. 1, with the balloon being shown in an extended configuration and in an inflated state;

FIG. 2B is a simplified schematic cross-sectional view illustration of the catheter system illustrated in FIG. 2A, with the balloon again being shown in the extended configuration, but with the balloon being in the deflated state;

FIG. 3A is a simplified schematic view illustration of a portion of an embodiment of the catheter system, including an embodiment of the balloon having features of the present invention, the balloon being shown in the inverted configuration; and

FIG. 3B is another simplified schematic view illustration of the portion of the catheter system illustrated in FIG. 3A, with the balloon being shown in the extended configuration.

While embodiments of the present invention are susceptible to various modifications and alternative forms, specifics thereof have been shown by way of example and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

Treatment of vascular lesions at treatment sites within a body of a patient can reduce major adverse events or death in affected subjects. As referred to herein, a major adverse event is one that can occur anywhere within the body due to the presence of a vascular lesion. Major adverse events can include, but are not limited to, major adverse cardiac events, major adverse events in the peripheral or central vasculature, major adverse events in the brain, major adverse events in the musculature, or major adverse events in any of the internal organs.

In various embodiments, the catheter systems and related methods disclosed herein can include a balloon catheter configured to advance to a vascular lesion, such as a calcified vascular lesion or a fibrous vascular lesion, at a treatment site located within a body of a patient. In certain implementations, the “treatment site” can be located at or near a vessel wall of a blood vessel of the patient. Additionally, or in the alternative, in other implementations, the “treatment site” can be at or near a heart valve of the patient. Further, or in the alternative, in still other implementations, the “treatment site” can be at another suitable location within the body of the patient.

As used herein, the terms “treatment site”, “intravascular lesion” and “vascular lesion” are used interchangeably unless otherwise noted. The intravascular lesions and/or the vascular lesions are sometimes referred to herein as “lesions”.

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings. The same or similar nomenclature and/or reference indicators will be used throughout the drawings, and the following detailed description to refer to the same or like parts.

In the interest of clarity, not all of the routine features of the implementations described herein are shown and described. It is appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is appreciated that such a development effort might be complex and time-consuming, but would nevertheless be a routine undertaking of engineering for those of ordinary skill in the art having the benefit of this disclosure.

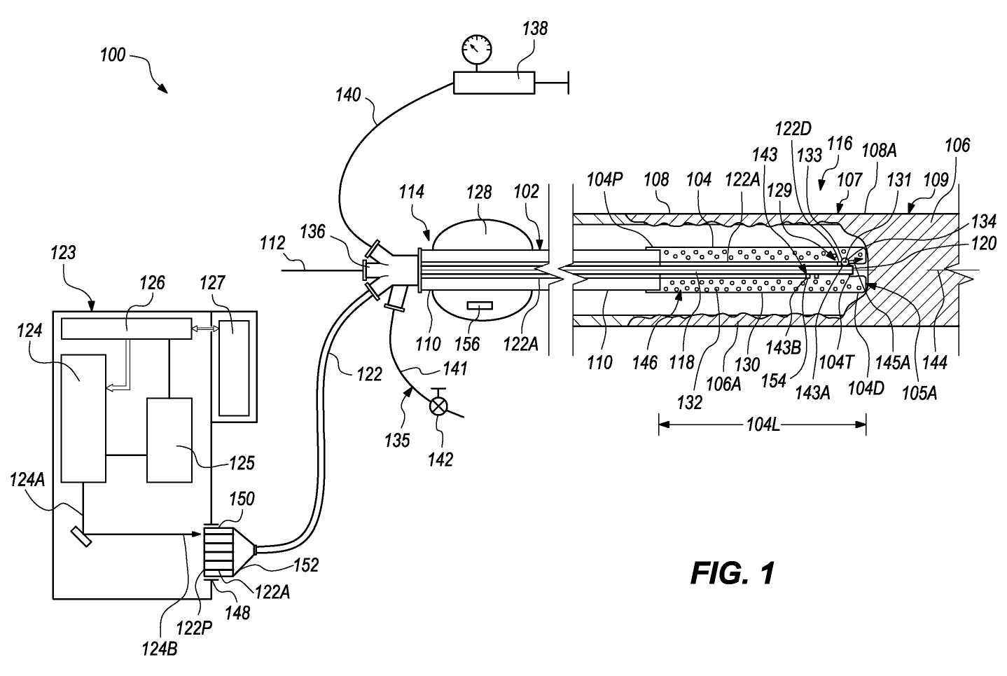

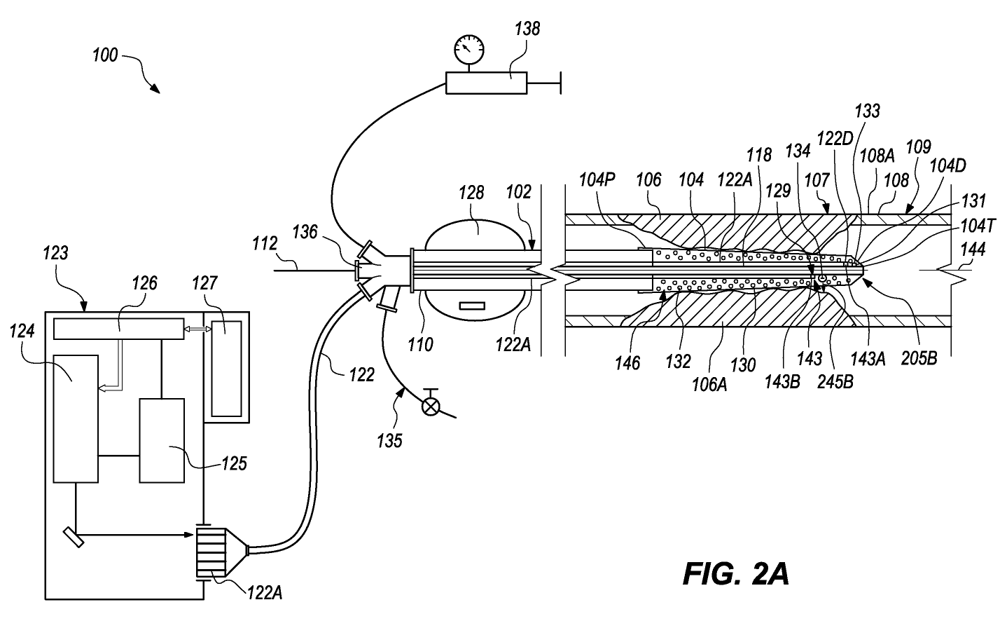

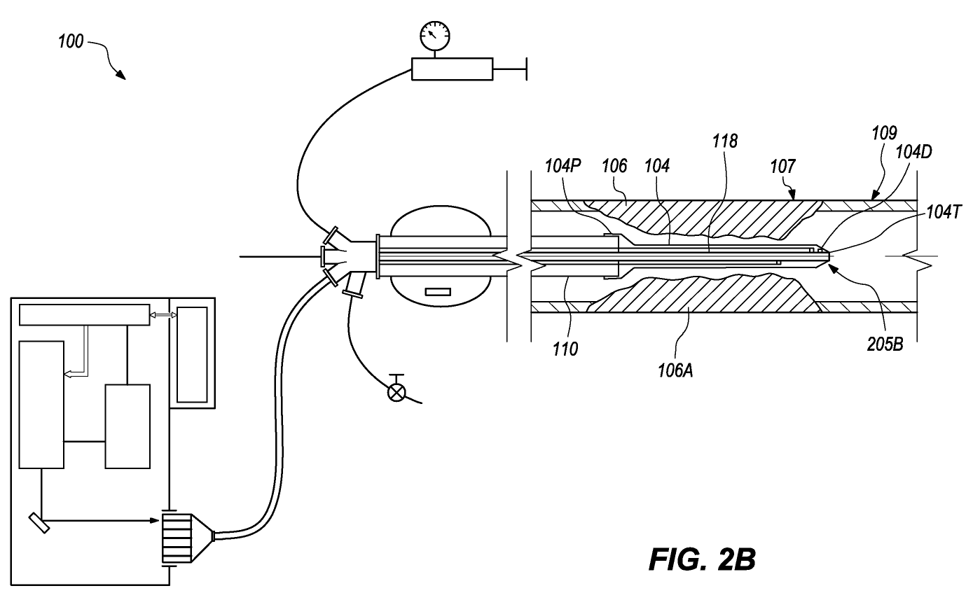

The catheter systems disclosed herein can include many different forms. Referring now to FIG. 1, a simplified schematic cross-sectional view illustration is shown of a catheter system 100 in accordance with various embodiments. The catheter system 100 is suitable for imparting acoustic waves and/or pressure waves to induce fractures within vascular lesions 106A at one or more treatment sites 106 within or adjacent to a vessel wall 108A of a blood vessel 108, or on or adjacent to a heart valve, within a body 107 of a patient 109. As described herein, in various embodiments, the catheter system 100 having features of the present invention can be useful in treating any types of vascular lesions 106A at the treatment site 106, but, in certain configurations, can be especially useful in addressing instances where the patient 109 is experiencing a chronic total occlusion (or “CTO”) of a blood vessel 108. Stated in another manner, in many embodiments, the catheter system 100 can be particularly useful when addressing treatment sites 106 that encompass and/or include a CTO. It is appreciated that in the implementation of the catheter system 100 illustrated in FIG. 1, the treatment site 106 and/or the vascular lesions 106A do encompass and/or include a CTO. However, it is further appreciated that the catheter system 100 can be used in alternative implementations, such as implementations in which the treatment site 106 and/or the vascular lesions 106A do not encompass and/or include a CTO. Such an alternative implementation is illustrated in FIG. 2A.

As shown in the embodiment illustrated in FIG. 1, the catheter system 100 can include (i) a catheter 102 including one or more of an inflatable balloon 104 (sometimes referred to herein as a “balloon”), a catheter shaft 110, a guidewire 112, a guidewire lumen 118, an energy guide bundle 122 including one or more energy guides 122A, a source manifold 136, a fluid pump 138, a handle assembly 128, an emitter assembly 129, and a fluid flushing system 135 (sometimes referred to herein as a “flushing system”) that is configured to selectively minimize contamination within the balloon 104; and (ii) a system console 123 including one or more of an energy source 124, a power source 125, a system controller 126, and a graphic user interface 127 (a “GUI”). In various embodiments, the emitter assembly 129 includes and/or incorporates at least one emitter station 143 and/or at least one emitter 131 that is configured to direct and/or concentrate energy toward one or more vascular lesions 106A at the treatment site 106 within or adjacent to the vessel wall 108A of the blood vessel 108, or within or adjacent to a heart valve, within the body 107 of the patient 109. Alternatively, the catheter system 100, the catheter 102 and/or the system console 123 can include more components or fewer components than those specifically illustrated and described in relation to FIG. 1.

As illustrated in FIG. 1, the catheter 102 is configured to move to the treatment site 106 within or adjacent to the vessel wall 108A of the blood vessel 108 within the body 107 of the patient 109, including instances in which the treatment site 106 encompasses and/or includes a CTO. The treatment site 106 can include one or more vascular lesions 106A such as calcified vascular lesions, for example. Additionally, or in the alternative, the treatment site 106 can include vascular lesions 106A such as fibrous vascular lesions. Still alternatively, in some implementations, the catheter 102 can be used at a treatment site 106 within or adjacent to a heart valve within the body 107 of the patient 109. Yet alternatively, in certain implementations, the catheter 102 can be used at a treatment site 106 at another suitable location within the body 107 of the patient 109.

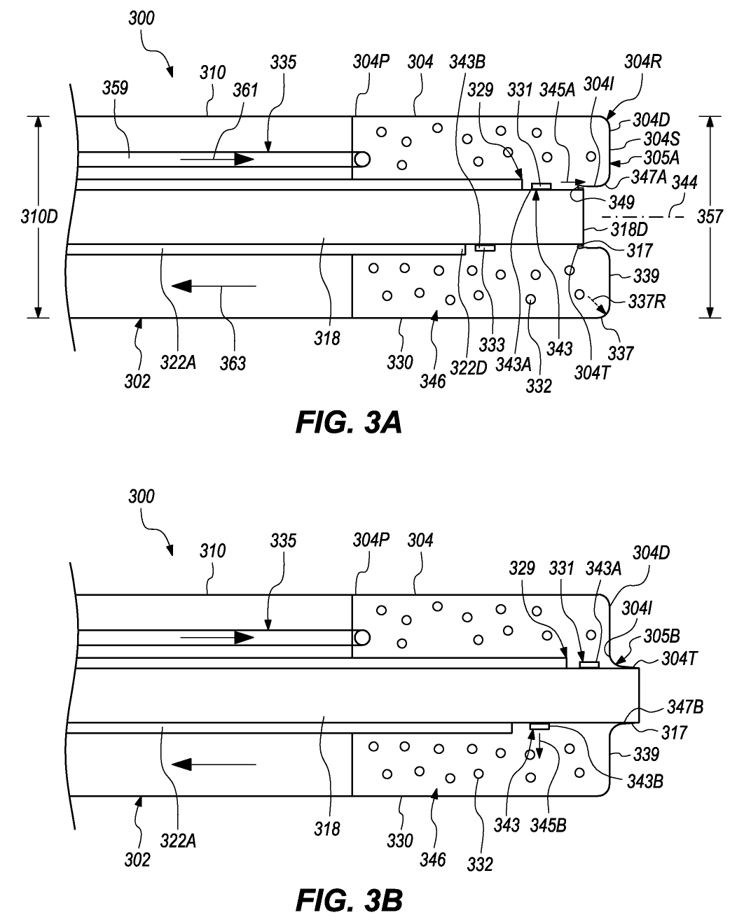

The catheter shaft 110 can extend from a proximal portion 114 of the catheter system 100 to a distal portion 116 of the catheter system 100. The catheter shaft 110 can include a longitudinal axis 144. The guidewire lumen 118 is configured to move over the guidewire 112. As utilized herein, the guidewire lumen 118 is substantially rigid and defines a conduit through which the guidewire 112 extends. The catheter shaft 110 and/or the catheter 102 can further include an inflation lumen 359 (illustrated, for example, in FIG. 3A), which is positioned at least partially within the catheter shaft 110 and which directs the catheter fluid 132 into the balloon interior 146, and/or various other lumens for various other purposes. In some embodiments, the catheter 102 and/or the guidewire lumen 118 can have a distal end opening 120 and can accommodate and be tracked over the guidewire 112 as the catheter 102 is moved and positioned at or near the treatment site 106.

The balloon 104 can include a balloon proximal end 104P and a balloon distal end 104D. In certain embodiments, the balloon 104 can be coupled to the catheter shaft 110 and/or to the guidewire lumen 118. More particularly, in some embodiments, the balloon proximal end 104P can be coupled and/or secured to the catheter shaft 110, and the balloon distal end 104D can be coupled and/or secured to the guidewire lumen 118.

The balloon 104 includes a balloon wall 130 that defines a balloon interior 146. The balloon 104 is selectively movable between a deflated state and an inflated state (as shown in FIG. 1). More specifically, the balloon 104 can be selectively inflated with a catheter fluid 132 (illustrated as a plurality of small circles) to move from the deflated state, suitable for advancing the catheter 102 through a patient’s vasculature, to the inflated state, suitable for anchoring the catheter 102 in position relative to the treatment site 106. Stated in another manner, when the balloon 104 is in the inflated state, at least a portion of the balloon wall 130 of the balloon 104 is configured to be positioned substantially directly adjacent to and/or in contact with the vascular lesions 106A at the treatment site 106.

As an overview, in various embodiments, the present invention is directed toward the balloon 104 and/or the balloon distal end 104D being selectively movable between a first (inverted) configuration 105A (as shown in FIG. 1) and a second (extended) configuration 205B (as shown in FIG. 2A). Stated in another manner, the balloon distal end 104D can be said to include a distal tip 104T that is selectively movable between being configured as an inverted tip, when the balloon 104 and/or the balloon distal end 104D is in the first (inverted) configuration 105A, and being configured as an extended tip, when the balloon 104 and/or the balloon distal end 104D is in the second (extended) configuration 205B.

More particularly, in many embodiments, with the balloon distal end 104D being coupled and/or secured to the guidewire lumen 118, selective, mechanical movement of the rigid guidewire lumen 118 relative to the catheter shaft 110 causes the balloon 104 to selectively move between the first (inverted) configuration 105A and the second (extended) configuration 205B. For example, in one movement, selective, mechanical movement of the rigid guidewire lumen 118 in a distal direction relative to the catheter shaft 110 can cause the balloon 104 to selectively move from the first (inverted) configuration 105A to the second (extended) configuration 205B. In another movement, selective, mechanical movement of the rigid guidewire lumen 118 in a proximal direction relative to the catheter shaft 110 can cause the balloon 104 to selectively move from the second (extended) configuration 205B to the first (inverted) configuration 105A. Alternatively, the balloon 104 can be selectively moved between the first (inverted) configuration 105A and the second (extended) configuration 205B in another suitable manner.

It is appreciated that the movement of the balloon 104 between the first (inverted) configuration 105A and the second (extended) configuration 205B can be reversed and repeated any suitable number of times and/or in any suitable manner. For example, the movement of guidewire lumen 118 relative to the catheter shaft 110 for purposes of moving the balloon 104 between the first (inverted) configuration 105A and the second (extended) configuration 205B can be repeated multiple times in close succession such that the guidewire lumen 118 is effectively moving back and forth relative to the catheter shaft 110, and the balloon 104 is moving back and forth between the first (inverted) configuration 105A and the second (extended) configuration 205B.

It is also appreciated that the selective, mechanical movement of the guidewire lumen 118 relative to the catheter shaft 110 for purposes of moving the balloon 104 between the first (inverted) configuration 105A and the second (extended) configuration 205B can be accomplished in any suitable manner, which can include both manual movements of the guidewire lumen 118 and automated movements of the guidewire lumen 118. For example, in one non-exclusive embodiment, the selective, mechanical movement of the guidewire lumen 118 relative to the catheter shaft 110 can be accomplished through use of a component, such as a configuration adjuster 155 of any suitable design, which can be positioned within the handle assembly 128. In such embodiment, the user or operator can simply manipulate the configuration adjuster 155 for purposes of selectively moving the guidewire lumen 118 relative to the catheter shaft 110 so that the balloon 104 is selectively moved between the first (inverted) configuration 105A and the second (extended) configuration 205B.

It is further appreciated that the configuration adjuster 155 can be configured to move the guidewire lumen 118 relative to the catheter shaft 110 any suitable distance for purposes of selectively moving the balloon 104 between the first (inverted) configuration 105A and the second (extended) configuration 205B. For example, in certain embodiments, the configuration adjuster 155 can move the guidewire lumen 118 between approximately 0.5 millimeters and ten millimeters relative to the catheter shaft 110 any suitable distance for purposes of selectively moving the balloon 104 between the first (inverted) configuration 105A and the second (extended) configuration 205B. In other embodiments, the configuration adjuster 155 can move the guidewire lumen 118 between approximately 0.5 millimeters and five millimeters relative to the catheter shaft 110 any suitable distance for purposes of selectively moving the balloon 104 between the first (inverted) configuration 105A and the second (extended) configuration 205B. In still other embodiments, the configuration adjuster 155 can move the guidewire lumen 118 between approximately one millimeter and two millimeters relative to the catheter shaft 110 any suitable distance for purposes of selectively moving the balloon 104 between the first (inverted) configuration 105A and the second (extended) configuration 205B. In yet other embodiments, the configuration adjuster 155 can move the guidewire lumen 118 another suitable distance relative to the catheter shaft 110 any suitable distance for purposes of selectively moving the balloon 104 between the first (inverted) configuration 105A and the second (extended) configuration 205B.

As referred to herein, the first (inverted) configuration 105A is a configuration in which at least a portion of the balloon 104 is positioned more distally than where the balloon distal end 104D and/or the distal tip 104T is coupled and/or secured to the guidewire lumen 118. Conversely, as referred to herein, the second (extended) configuration is a configuration in which no portion of the balloon 104 is positioned more distally than where the balloon distal end 104D and/or the distal tip 104T is coupled and/or secured to the guidewire lumen 118. As described in detail herein, in many implementations, the different configurations 105A, 205B for the balloon 104 can be utilized for treating different types of lesions 106A and/or different types of treatment sites 106.

In certain embodiments, when the balloon 104 is in the first (inverted) configuration 105A, the balloon 104 and/or the balloon distal end 104D can have a shape that enables a greater surface area of the balloon 104 to be positioned substantially directly adjacent to a CTO that can be encompassed and/or included within the treatment site 106. For example, in many embodiments, when the balloon 104 is in the first (inverted) configuration 105A, the balloon 104 can have a bullnose shape at the balloon distal end 104D. As referred to herein, a “bullnose shape” for the balloon distal end 104D of the balloon 104 is intended to signify the balloon distal end 104D having a rounded, forward perimeter 337 (illustrated, for example, in FIG. 3A), with a particular radius of curvature 337R (illustrated, for example, in FIG. 3A), while still allowing for the balloon distal end 104D to be at least somewhat or generally flat as it extends radially inwardly from the rounded, forward perimeter 337. More particularly, in several embodiments, the bullnose-shaped balloon distal end 104D includes the balloon distal end 104D having the rounded, forward perimeter 337 and a substantially flat portion 339 (illustrated, for example, in FIG. 3A) that extends radially inwardly therefrom.

As described in greater detail herein below, in some embodiments, it can be desired that the radius of curvature 337R be relatively small in order to better enable a larger surface area of the balloon 104, such as the flat portion 339 of the balloon distal end 104D, to be positioned substantially directly adjacent to the CTO that can be encompassed and/or included within the treatment site 106. More particularly, a smaller radius of curvature 337R for the rounded, forward perimeter 337 of the balloon 104, in combination with a corresponding larger flat portion 339 that extends radially inwardly therefrom, at the balloon distal end 104D helps to maximize the surface area of the balloon 104 that can be positioned substantially directly adjacent to the treatment site 106.

In some embodiments, when the balloon distal end 104D is in the first (inverted) configuration 105A, the balloon 104 further includes an inverted region 347A (illustrated in FIG. 3A) that extends proximally from the flat portion 339 for purposes of effectively coupling and/or bonding the balloon distal end 104D and/or the distal tip 104T to the guidewire lumen 118, which, when the balloon 104 is in the first (inverted) configuration 105A, does not extend as far distally as a portion of the balloon 104 at or near the balloon distal end 104D. It is appreciated that the bullnose shape and/or the first (inverted) configuration 105A for the balloon distal end 104D helps to provide improved delivery of the acoustic energy waves and/or pressure waves to the treatment site 106, which, as noted, can include the CTO. It is further appreciated that the design of the balloon 104, as described in detail herein, allows the emitter stations 143 and/or the emitters 131 within the balloon interior 146 to be positioned closer to the treatment site 106 and/or the CTO than other currently existing intravascular lithotripsy devices.

In certain embodiments, when the balloon distal end 104D is in the second (extended) configuration 205B, the balloon 104 no longer includes the inverted region 347A, but rather the same portion of the balloon 104 can be configured as an extended region 347B (illustrated in FIG. 3B) that extends distally from the flat portion 339 while still enabling the effective coupling and/or bonding of the balloon distal end 104D and/or the distal tip 104T to the guidewire lumen 118. It is appreciated that when the balloon 104 is in the second (extended) configuration 205B, the catheter system 100 can be better configured to treat vascular lesions 106A at the treatment site 106 that are positioned more radially within the blood vessel 108 and do not necessarily include a CTO.

As described herein, it is further appreciated that individual emitter stations 143 and/or individual emitters 131 can also be configured to better treat certain types of vascular lesions 106A at the treatment site 106, with or without a CTO, regardless of whether the balloon 104 is in the first (inverted) configuration 105A or the second (extended) configuration 205B.

In some embodiments, the balloon distal end 104D and/or the distal tip 104T can have a tapered design when the balloon 104 is in the second (extended) configuration 205B and the balloon 104 is in the deflated state. More particularly, when the balloon 104 is in the deflated state for purposes of positioning the catheter 102 within the body 107 of the patient 109, it can be preferred to have the balloon 104 in the second (extended) configuration 205B to enhance the crossing profile as the balloon 104 engages the vascular lesions 106A at the treatment site 106, and to provide a tapered distal tip 104T for better crossing. Subsequently, once the balloon 104 is through the vascular lesions 106A and/or is positioned as desired at the treatment site 106, the balloon 104 can be moved to the inflated state (the balloon 104 can be selectively inflated with the catheter fluid 132). The balloon 104 can then further be used for performing the desired treatment of the vascular lesions 106A in the second (extended) configuration 205B or the first (inverted) configuration 105A. It is appreciated that if it is desired to use the balloon 104 in the first (inverted) configuration 105A, such as in the treatment of vascular lesions 106A in the form of a CTO, it would be necessary to move the balloon 104 from the second (extended) configuration 205B to the first (inverted) configuration 105A in any suitable manner such as described in detail herein.

Embodiments of the balloon 104 that enable the balloon distal end 104D and/or the distal tip 104T to remain effectively coupled and/or bonded to the guidewire lumen 118 while moving between the first (inverted) configuration 105A and the second (extended) configuration 205B will be illustrated and described in greater detail herein below.

As provided herein, in various embodiments, the catheter system 100 and/or the emitter assembly 129 can include the at least one emitter station 143 and/or the at least one emitter 131 that is configured to transmit energy from the energy source 124 into the balloon interior 146 in order to generate plasma and/or pressure waves in the catheter fluid 132 within the balloon interior 146. As referred to herein, each emitter station 143 can include one or more emitters 131 that are positioned at approximately the same longitudinal position within the balloon interior 146 relative to a balloon length 104L of the balloon 104.

Each of the emitters 131 includes a guide distal end 122D of one of the energy guides 122A, which is positioned within the balloon interior 146, and a corresponding plasma target 133 (also sometimes referred to as a “plasma generating structure” or a “plasma generator”) that is positioned near, but typically spaced apart from, the guide distal end 122D. As so defined, the emitters 131, and/or the components thereof, are configured to direct and/or concentrate energy generated in the catheter fluid 132 within the balloon interior 146 so as to impart pressure onto and induce fractures in the vascular lesions 106A at the treatment site 106. More specifically, during use of the catheter system 100, energy from the energy source 124 is directed toward and received by the energy guide 122A, is guided through the energy guide 122A, and is then emitted from at or near the guide distal end 122D of the energy guide 122A. The energy emitted from at or near the guide distal end 122D is directed toward and contacts and energizes the corresponding plasma target 133 for purposes of generating the plasma in the catheter fluid 132 within the balloon interior 146.

As referred to herein, the plasma target 133 or “plasma generator” can include and/or incorporate any suitable type of structure and can be positioned and oriented in any suitable manner at or near the guide distal end 122D of the energy guide 122A. For example, in certain embodiments, the plasma target 133 can be configured, positioned and/or oriented such that the emitter 131 operates as a forward-firing emitter, which can enable the emitters 131 to more effectively deliver the acoustic energy waves and/or pressure waves directly toward the CTO at the treatment site 106. As referred to herein, a “forward-firing” emitter is one that fires in a direction that is substantially parallel to the longitudinal axis 144 of the catheter shaft 110.

Alternatively, the plasma target 133 can have another suitable structural design and/or can be positioned and oriented in another suitable manner. In other embodiments, the emitter 131 can be configured as other than a forward-firing emitter. For example, in some alternative embodiments, the emitter 131 can be configured as a lateral-firing emitter that fires more in a radial direction toward the balloon wall 130 of the balloon 104. More particularly, when the emitters 131 are lateral-firing emitters, the emitters 131 fire in a direction that is generally transverse, or at a suitable angle, relative to the longitudinal axis 144 of the catheter shaft 110. It is appreciated that the lateral-firing emitters can be more effective at treating vascular lesions 106A that are distributed more radially about the blood vessel 108, but that do not include or encompass a CTO.

In many embodiments, with the first (inverted) configuration 105A for the balloon distal end 104D being better suited for treating treatment sites 106 that include a CTO, and with the second (extended) configuration 205B for the balloon distal end 104D being better suited for treating treatment sites 106 with more radially distributed lesions 106A (non-CTO), the catheter system 100 can include a mix of both forward-firing emitters and lateral-firing emitters. For example, in certain non-exclusive alternative embodiments, the catheter system 100 can include a first emitter station 143A at a first longitudinal position within the balloon interior 146 that is nearer to the balloon distal end 104D, which can include at least one emitter 131 that is configured to operate as a forward-firing emitter; and a second emitter station 143B at a second longitudinal position within the balloon interior 146 that is farther from the balloon distal end 104D than the first emitter station 143A, which can include at least one emitter 131 that is configured to operate as a lateral-firing (non-forward-firing) emitter.

In the implementation shown in FIG. 1, because the treatment site 106 includes a CTO, the first emitter station 143A with the forward-firing emitter 131 is shown to be active and firing, while the second emitter station 143B with the lateral-firing emitter 131 is shown to be not active and not firing.

The balloon 104 suitable for use in the catheter system 100 includes those that can be passed through the vasculature of a patient 109 when in the deflated state. In some embodiments, the balloon 104 is made from silicone. In other embodiments, the balloon 104 can be made from materials such as polydimethylsiloxane (PDMS), polyurethane, polymers such as a polyether block amide (such as PEBAX™) material, nylon, or any other suitable material.

The balloon 104 can have any suitable resilience for purposes of providing and enabling the desired and/or necessary force to be imparted to the vascular lesions 106A at the treatment site 106. For example, in some embodiments, the balloon 104 can be formed from a non-resilient material. In other embodiments, the balloon 104 can be formed from a semi-resilient material. In still other embodiments, the balloon 104 can be formed from a resilient material.

The balloon 104 can have any suitable inflated balloon diameter (in the inflated state). In various embodiments, the balloon 104 can have an inflated balloon diameter ranging from less than 0.5 millimeters (mm) up to 25 mm. In some embodiments, the balloon 104 can have an inflated balloon diameter ranging from at least one mm up to 14 mm. In certain embodiments, the balloon 104 can have an inflated balloon diameter ranging from at least one mm up to two mm. Alternatively, the balloon 104 can have an inflated balloon diameter that is greater than or less than the ranges specifically noted herein.

In some embodiments, the balloon 104 can have a balloon diameter that is substantially equal to a shaft diameter of the catheter shaft 110. In certain such embodiments, the balloon 104 can be formed from a non-resilient material such that the balloon diameter of the balloon 104 is substantially equal to the shaft diameter of the catheter shaft 110 regardless of whether the balloon 104 is in the inflated state or the deflated state. Moreover, with the use of non-resilient material for the balloon 104, the balloon diameter will be substantially the same regardless of whether the balloon 104 is in the inflated state or the deflated state. Stated in another manner, in such embodiments, the inflated diameter of the balloon 104 will be substantially equal to a deflated diameter of the balloon 104.

It is appreciated that in embodiments in which the balloon diameter is substantially equal to the shaft diameter, even when the balloon is in the inflated state, then the shaft diameter could also range from less than 0.5 mm up to 25 mm, from at least one mm up to 14 mm, from at least one mm up to two mm, or another suitable range.

In some embodiments, the balloon 104 can have a balloon length 104L ranging from at least three mm to 300 mm. More particularly, in certain embodiments, the balloon 104 can have a balloon length 104L ranging from at least eight mm to 200 mm. Alternatively, the balloon 104 can have a balloon length 104L that is different than the specific ranges noted herein.

It is appreciated that, in many embodiments, regardless of whether the balloon 104 is formed from a non-resilient material, a semi-resilient material or a resilient material, the balloon length 143 of the balloon 104 can remain substantially constant during movement between the deflated state and the inflated state. Stated in another manner, regardless of the resilience of the material used to form the balloon 104, the balloon distal end 104D does not expand distally and the balloon proximal end 104P does not expand proximally during movement from the deflated state to the inflated state.

It is further noted, however, that during movement of the balloon distal end 104D between the first (inverted) configuration 105A and the second (extended) configuration 205B, the balloon length 104L will change minimally simply due to the movement of the distal tip 104T. Stated in another manner, the balloon distal end 104D and/or the distal tip 104T will extend slightly more distally when the balloon distal end 104D is in the second (extended) configuration 205B as compared to when the balloon distal end 104D is in the first (inverted) configuration 105A. However, during such movement between configurations 105A, 205B, the general overall structure of the balloon 104 will not extend and/or retract so as to otherwise impact the balloon length 104L of the balloon 104.

In certain embodiments, the balloon 104 can be configured such that the balloon 104 is not tapered anywhere along the balloon length 104L of the balloon 104 from the balloon proximal end 104P to the balloon distal end 104D. Alternatively, the balloon 104 can include one or more tapered portions along the balloon length 104L of the balloon 104 from the balloon proximal end 104P to the balloon distal end 104D. It is appreciated, however, that whether or not the balloon 104 includes any tapered portions along the balloon length 104L can be different when the balloon distal end 104D is in the first (inverted) configuration 105A versus when the balloon distal end 104D is in the second (extended) configuration 205B. For example, in one non-exclusive embodiment, the balloon 104 can have no tapered portions along its full balloon length 104L when the balloon distal end 104D is in the first (inverted) configuration 105A, but the balloon distal end 104D can include a tapered portion, such as substantially adjacent to the balloon distal end 104D, after the balloon distal end 104D has been moved to the second (extended) configuration 205B. It is further appreciated, however, that, in another non- exclusive embodiment, the balloon 104 may still not include any tapered portions even after the balloon distal end 104D has been moved to the second (extended) configuration 205B.

The balloon 104 can be inflated to inflation pressures of between approximately one atmosphere (atm) and 70 atm. In some embodiments, the balloon 104 can be inflated to inflation pressures of from at least 20 atm to 60 atm. In other embodiments, the balloon 104 can be inflated to inflation pressures of from at least six atm to 20 atm. In still other embodiments, the balloon 104 can be inflated to inflation pressures of from at least three atm to 20 atm. In yet other embodiments, the balloon 104 can be inflated to inflation pressures of from at least two atm to ten atm.

The balloon 104 can have various shapes. In various embodiments, as noted above, the balloon 104 can be designed such that the balloon distal end 104D has a bullnose shape or bullnose design, at least when the balloon distal end 104D is in the first (inverted) configuration 105A, that is configured to be positioned substantially adjacent to a chronic total occlusion included at the treatment site 106 within the blood vessel 108 of the patient 109. It is appreciated that the overall shape of the balloon 104 can be somewhat varied as the balloon distal end 104D is moved between the first (inverted) configuration 105A and the second (extended) configuration 205B. For example, it is appreciated that the balloon distal end 104D may no longer exhibit the same bullnose shape after the balloon distal end 104D has been moved to the second (extended) configuration 205B.

In some embodiments, as further noted above, the balloon 104 can have no taper, and can have an inverted balloon bond, such as when the balloon distal end 104D is in the first (inverted) configuration 105A, for purposes of coupling the balloon distal end 104D and/or the distal tip 104T of the balloon 104 to the guidewire lumen 118, which is configured to maximize acoustic energy that is imparted to the chronic total occlusion (CTO). As also noted above, with such design, the emitters 131 can be positioned near the balloon distal end 104D such that the emitters 131 within the balloon interior 146 are positioned closer to the treatment site 106 and/or the CTO than other currently existing intravascular lithotripsy devices. In many embodiments, as illustrated and described in greater detail herein below, the emitters 131 are positioned proximally relative to the forward perimeter 337 of the balloon 104. Stated in another manner, in such embodiments, the guide distal end 122D of the energy guide 122A and the corresponding plasma generator 133 that cooperate to form the emitter 131 are positioned more proximally than the rounded, forward perimeter 337 at or near the balloon distal end 104D of the balloon 104.

Additionally, or in the alternative, in other embodiments, the balloon 104 can have a conical shape, a square shape, a rectangular shape, a spherical shape, a conical/square shape, a conical/spherical shape, an extended spherical shape, an oval shape, a tapered shape, a bone shape, a stepped diameter shape, an offset shape, a conical offset shape, or another suitable shape.

In some embodiments, the balloon 104 can include a drug eluting coating or a drug eluting stent structure. The drug eluting coating or drug eluting stent can include one or more therapeutic agents including anti-inflammatory agents, anti-neoplastic agents, anti-angiogenic agents, and the like. The drug eluting coating or drug eluting stent can be utilized to further treatment of the treatment site 106 before, during or after any given intravascular lithotripsy procedure.

The catheter fluid 132 used to inflate the balloon 104 can be a liquid or a gas. Some examples of the catheter fluid 132 suitable for use can include, but are not limited to one or more of water, saline, contrast medium, fluorocarbons, perfluorocarbons, gases, such as carbon dioxide, or any other suitable catheter fluid 132. In some embodiments, the catheter fluid 132 can be used as a base inflation fluid. In some embodiments, the catheter fluid 132 can include a mixture of saline to contrast medium in a volume ratio of approximately 50:50. In other embodiments, the catheter fluid 132 can include a mixture of saline to contrast medium in a volume ratio of approximately 25:75. In still other embodiments, the catheter fluid 132 can include a mixture of saline to contrast medium in a volume ratio of approximately 75:25. However, it is understood that any suitable ratio of saline to contrast medium can be used. The catheter fluid 132 can be tailored on the basis of composition, viscosity, and the like so that the generation of plasma and the rate of travel of the acoustic waves and/or pressure waves are appropriately manipulated. In certain embodiments, the catheter fluid 132 suitable for use herein is biocompatible. A volume of catheter fluid 132 can be tailored by the chosen energy source 124 and the type of catheter fluid 132 used.

In certain embodiments, the catheter fluid 132 can include a wetting agent or surface-active agent (surfactant). These compounds can lower the tension between solid and liquid matter. These compounds can act as emulsifiers, dispersants, detergents, and water infiltration agents. Wetting agents or surfactants reduce surface tension of the liquid and allow it to fully wet and come into contact with optical components (such as the energy guide(s) 122A) and mechanical components (such as other portions of the emitter assembly 129). This reduces or eliminates the accumulation of bubbles and pockets or inclusions of gas within the emitter assembly 129. Nonexclusive examples of chemicals that can be used as wetting agents include, but are not limited to, benzalkonium chloride, benzethonium chloride, cetylpyridinium chloride, Poloxamer 188, Poloxamer 407, Polysorbate 20, Polysorbate 40, and the like. Non-exclusive examples of surfactants can include, but are not limited to, ionic and non-ionic detergents, and sodium stearate. Another suitable surfactant is 4-(5-dodecyl) benzenesulfonate. Other examples can include docusate (dioctyl sodium sulfosuccinate), alkyl ether phosphates, and perfluorooctanesulfonate (PFOS), to name a few.

By using a wetting agent or surfactant, direct liquid contact with the energy guide 122A allows the energy to be more efficiently converted into a plasma. Using the wetting agent or surfactant with the small dimensions of the optical and mechanical components used in the emitter assembly 129 and other parts of the catheter 102, it is less difficult to achieve greater (or complete) wetting. Decreasing the surface tension of the liquid can decrease the difficulty for such small structures to be effectively wetted by the liquid and therefore be nearly or completely immersed. By reducing or eliminating air or other gas bubbles from adhering to the optical and mechanical structure and energy guides 122A, considerable increase in efficiency of the device can occur.

The specific percentage of the wetting agent or surfactant can be varied to suit the design parameters of the catheter system 100 and/or the emitter assembly 129 being used. In one embodiment, the percentage of the wetting agent or surfactant can be less than approximately 50% by volume of the catheter fluid 132. In non-exclusive alternative embodiments, the percentage of the wetting agent or surfactant can be less than approximately 40%, 30%, 20%, 10%, 5%, 2%, 1%, 0.1% or 0.01% by volume of the catheter fluid 132. Still alternatively, the percentage of the wetting agent or surfactant can fall outside of the foregoing ranges.

In some embodiments, the contrast agents used in the contrast media can include, but are not to be limited to, iodine-based contrast agents, such as ionic or non- ionic iodine-based contrast agents. Some non-limiting examples of ionic iodine-based contrast agents include diatrizoate, metrizoate, iothalamate, and ioxaglate. Some non- limiting examples of non-ionic iodine-based contrast agents include iopamidol, iohexol, ioxilan, iopromide, iodixanol, and ioversol. In other embodiments, non-iodine-based contrast agents can be used. Suitable non-iodine containing contrast agents can include gadolinium (III)-based contrast agents. Suitable fluorocarbon and perfluorocarbon agents can include, but are not to be limited to, agents such as the perfluorocarbon dodecafluoropentane (DDFP, C₅F₁₂).

The catheter fluids 132 can include those that include absorptive agents that can selectively absorb light in the ultraviolet region (e.g., at least ten nanometers (nm) to 400 nm), the visible region (e.g., at least 400 nm to 780 nm), or the near-infrared region (e.g., at least 780 nm to 2.5 µm) of the electromagnetic spectrum. Suitable absorptive agents can include those with absorption maxima along the spectrum from at least ten nm to 2.5 µm. Alternatively, the catheter fluids 132 can include those that include absorptive agents that can selectively absorb light in the mid-infrared region (e.g., at least 2.5 µm to 15 µm), or the far-infrared region (e.g., at least 15 µm to one mm) of the electromagnetic spectrum. In various embodiments, the absorptive agent can be those that have an absorption maximum matched with the emission maximum of the laser used in the catheter system 100. By way of non-limiting examples, various lasers usable in the catheter system 100 can include neodymium:yttrium-aluminum-garnet (Nd:YAG – emission maximum = 1064 nm) lasers, holmium:YAG (Ho:YAG – emission maximum = 2.1 µm) lasers, or erbium:YAG (Er:YAG – emission maximum = 2.94 µm) lasers. In some embodiments, the absorptive agents can be water soluble. In other embodiments, the absorptive agents are not water soluble. In some embodiments, the absorptive agents used in the catheter fluids 132 can be tailored to match the peak emission of the energy source 124. Various energy sources 124 having emission wavelengths of at least ten nanometers to one millimeter are discussed elsewhere herein.

The catheter shaft 110 of the catheter 102 can be coupled to the plurality of energy guides 122A of the energy guide bundle 122 that are in optical communication with the energy source 124. The energy guide(s) 122A can be disposed along the catheter shaft 110 and within the balloon 104. Each of the energy guides 122A can have the guide distal end 122D that is at any suitable longitudinal position relative to the balloon length 104L of the balloon 104 and/or relative to a length of the guidewire lumen 118. Stated in another manner, the guide distal end 122D of each of the energy guides 122A can be positioned at or within any particular emitter station 143 along with the corresponding plasma target 133 that cooperate to form the individual emitter 131.

In many embodiments, each energy guide 122A can be an optical fiber and the energy source 124 can be a light source such as a laser. The energy source 124 can be in optical communication with the energy guides 122A at the proximal portion 114 of the catheter system 100. More particularly, the energy source 124 can selectively, simultaneously, sequentially and/or alternatively be in optical communication with each of the energy guides 122A in any desired combination, sequence and/or pattern.

In some embodiments, the catheter shaft 110 can be coupled to multiple energy guides 122A such as a first energy guide, a second energy guide, a third energy guide, etc., which can be disposed at any suitable positions about and/or relative to the guidewire lumen 118 and/or the catheter shaft 110. For example, in certain non-exclusive embodiments, two energy guides 122A can be spaced apart from one another by approximately 180 degrees about the circumference of the guidewire lumen 118 and/or the catheter shaft 110; three energy guides 122A can be spaced apart from one another by approximately 120 degrees about the circumference of the guidewire lumen 118 and/or the catheter shaft 110; four energy guides 122A can be spaced apart from one another by approximately 90 degrees about the circumference of the guidewire lumen 118 and/or the catheter shaft 110; five energy guides 122A can be spaced apart from one another by approximately 72 degrees about the circumference of the guidewire lumen 118 and/or the catheter shaft 110; six energy guides 122A can be spaced apart from one another by approximately 60 degrees about the circumference of the guidewire lumen 118 and/or the catheter shaft 110; eight energy guides 122A can be spaced apart from one another by approximately 45 degrees about the circumference of the guidewire lumen 118 and/or the catheter shaft 110; or ten energy guides 122A can be spaced apart from one another by approximately 36 degrees about the circumference of the guidewire lumen 118 and/or the catheter shaft 110. Still alternatively, multiple energy guides 122A need not be uniformly spaced apart from one another about the circumference of the guidewire lumen 118 and/or the catheter shaft 110. More particularly, it is further appreciated that the energy guides 122A can be disposed uniformly or non-uniformly about the guidewire lumen 118 and/or the catheter shaft 110 to achieve the desired effect in the desired locations.

In certain embodiments, the guidewire lumen 118 can be substantially annular- shaped and/or cylindrical-shaped and can include grooves formed along and/or into an outer surface of the guidewire lumen 118, with the grooves extending in a generally longitudinal direction along the guidewire lumen 118. In such embodiments, each of the energy guides 122A and/or the emitter(s) 131 of the emitter assembly 129 can be positioned, received and retained within an individual groove formed along and/or into the outer surface of the guidewire lumen 118. Alternatively, the guidewire lumen 118 can be formed without a grooved outer surface, and the position of the energy guides 122A and/or the emitter(s) 131 of the emitter assembly 129 relative to the guidewire lumen 118 can be maintained in another suitable manner.

The catheter system 100, the catheter 102 and/or the energy guide bundle 122 can include any number of energy guides 122A in optical communication with the energy source 124 at the proximal portion 114, and with the catheter fluid 132 within the balloon interior 146 of the balloon 104 at the distal portion 116. For example, in some embodiments, the catheter system 100, the catheter 102 and/or the energy guide bundle 122 can include from one energy guide 122A to greater than 30 energy guides 122A.

The guide distal end 122D of each of the energy guides 122A, and/or the emitters 131 as a whole, can be at any suitable or desired longitudinal position within the balloon interior 146 relative to the balloon length 104L of the balloon 104. It is appreciated that more than one guide distal end 122D and/or emitter 131 can be at any suitable or desired longitudinal position within the balloon interior 146 relative to the balloon length 104L. In many embodiments, the guide distal end 122D of one or more energy guides 122A, and/or one or more emitters 131, can be at any given longitudinal position relative to the balloon length 104L of the balloon 104 and/or relative to a length of the guidewire lumen 118. As noted above, each such longitudinal position that includes the guide distal end 122D of one or more energy guides 122A, and/or includes one or more emitters 131, can be referred to as an emitter station 143. Because the guide distal end 122D of the energy guide 122A and the corresponding plasma target 133 are positioned so close together, the emitter 131 that encompasses the guide distal end 122D and the corresponding plasma target 133, and thus each emitter station 143, can be referred to as being positioned at a single (approximate) longitudinal position within the balloon interior 146. Each emitter station 143 can be located and/or configured to treat different treatment sites 106 and/or different particular areas within any given treatment site 106. Thus, as so described, the catheter system 100, the catheter 102 and/or the energy guide bundle 122 can also include any suitable number of emitter stations 143.

In certain embodiments, a distal-most emitter station 143 (such as the first emitter station 143A) can include one or more emitters 131 that are configured to operate as forward-firing emitters 131; and each additional emitter station 143 (such as the second emitter station 143B) can include one or more emitters 131 that are configured to operate as other than forward-firing emitters 131. Stated in another manner, in such embodiments, the distal-most emitter station 143 can include forward-firing emitters 131 that are configured to more effectively treat a CTO that may be encompassed and/or included within the treatment site 106; and each additional emitter station 143 can include emitters 131 that are lateral-firing emitters 131, or emitters 131 that fire at any other suitable angle that is not substantially parallel to the longitudinal axis 144 of the catheter shaft 110.

The energy guides 122A can have any suitable design that is useful and appropriate for purposes of enabling the generation of plasma, acoustic waves and/or pressure waves in the catheter fluid 132 within the balloon interior 146. Thus, the general description of the energy guides 122A as light guides is not intended to be limiting in any manner, except for as set forth in the claims appended hereto. More particularly, although the catheter systems 100 are often described with the energy source 124 as a light source and the one or more energy guides 122A as light guides, the catheter system 100 can alternatively include any suitable energy source 124 and energy guides 122A for purposes of enabling the generation of the desired plasma in the catheter fluid 132 within the balloon interior 146. For example, in one non-exclusive alternative embodiment, the energy source 124 can be configured to provide high voltage pulses, and each energy guide 122A can include an electrode pair including spaced apart electrodes that extend into the balloon interior 146. In such embodiment, each pulse of high voltage is applied to the electrodes and forms an electrical arc across the electrodes, which, in turn, generates the plasma and forms the acoustic waves and/or pressure waves in the catheter fluid 132 that are utilized to provide the fracture force onto the vascular lesions 106A at the treatment site 106. Still alternatively, the energy source 124 and/or the energy guides 122A can have another suitable design and/or configuration, be it electrical, acoustic, pneumatic, other mechanical, etc.

In certain embodiments, the energy guides 122A can include an optical fiber or flexible light pipe. The energy guides 122A can be thin and flexible and can allow light signals to be sent with very little loss of strength. The energy guides 122A can include a core surrounded by a cladding about its circumference. In some embodiments, the core can be a cylindrical core or a partially cylindrical core. The core and cladding of the energy guides 122A can be formed from one or more materials, including but not limited to one or more types of glass, silica, or one or more polymers. The energy guides 122A may also include a protective coating, such as a polymer. It is appreciated that the index of refraction of the core will be greater than the index of refraction of the cladding.

Each energy guide 122A can guide energy along its length from a guide proximal end 122P toward the guide distal end 122D, with the guide distal end 122D having at least one optical window (not shown) that is positioned within the balloon interior 146.

The energy guides 122A can assume many configurations about and/or relative to the catheter shaft 110 of the catheter 102. In some embodiments, the energy guides 122A can run parallel to the longitudinal axis 144 of the catheter shaft 110. In some embodiments, the energy guides 122A can be physically coupled to the catheter shaft 110. In other embodiments, the energy guides 122A can be disposed along a length of an outer diameter of the catheter shaft 110. In yet other embodiments, the energy guides 122A can be disposed within one or more energy guide lumens within the catheter shaft 110.

The energy guides 122A can also be disposed at any suitable positions about the circumference of the guidewire lumen 118 and/or the catheter shaft 110, and the guide distal end 122D of each of the energy guides 122A can be disposed at any suitable longitudinal position relative to the balloon length 104L of the balloon 104 and/or relative to the length of the guidewire lumen 118 to more effectively and more precisely impart acoustic waves and/or pressure waves for purposes of disrupting the vascular lesions 106A at the treatment site 106.

In certain embodiments, the energy guides 122A can include one or more photoacoustic transducers 154, where each photoacoustic transducer 154 can be in optical communication with the energy guide 122A within which it is disposed. In some embodiments, the photoacoustic transducers 154 can be in optical communication with the guide distal end 122D of the energy guide 122A. In such embodiments, the photoacoustic transducers 154 can have a shape that corresponds with and/or conforms to the guide distal end 122D of the energy guide 122A.

The photoacoustic transducer 154 is configured to convert light energy into an acoustic wave at or near the guide distal end 122D of the energy guide 122A. The direction of the acoustic wave can be tailored by changing an angle of the guide distal end 122D of the energy guide 122A.

In certain embodiments, the photoacoustic transducers 154 disposed at the guide distal end 122D of the energy guide 122A can assume the same shape as the guide distal end 122D of the energy guide 122A. For example, in certain non-exclusive embodiments, the photoacoustic transducer 154 and/or the guide distal end 122D can have a conical shape, a convex shape, a concave shape, a bulbous shape, a square shape, a stepped shape, a half-circle shape, an ovoid shape, and the like. The energy guide 122A can further include additional photoacoustic transducers 154 disposed along one or more side surfaces along the length of the energy guide 122A.

In some embodiments, the energy guides 122A and/or the emitter assembly 129 can further include one or more diverting structures or “diverters” (not shown in FIG. 1), such as within the energy guide 122A and/or near the guide distal end 122D of the energy guide 122A, that are configured to direct energy from the energy guide 122A toward a side surface which can be located at or near the guide distal end 122D of the energy guide 122A, before the energy is directed toward the balloon wall 130. A diverting structure can include any structure of the system that diverts energy from the energy guide 122A away from its axial path toward a side surface of the energy guide 122A. The energy guides 122A can each include one or more optical windows disposed along the longitudinal or circumferential surfaces of each energy guide 122A and in optical communication with a diverting structure. Stated in another manner, the diverting structures can have any suitable structural configuration that is configured to direct energy in the energy guide 122A toward a side surface that is at or near the guide distal end 122D, where the side surface is in optical communication with an optical window. The optical windows can include a portion of the energy guide 122A that allows energy to exit the energy guide 122A from within the energy guide 122A, such as a portion of the energy guide 122A lacking a cladding material on or about the energy guide 122A.

Examples of the diverting structures suitable for use include a reflecting element, a refracting element, and a fiber diffuser. The diverting structures suitable for focusing energy away from the tip of the energy guides 122A can include, but are not to be limited to, those having a convex surface, a gradient-index (GRIN) lens, and a mirror focus lens. Upon contact with the diverting structure, the energy can be diverted within the energy guide 122A to one or more of the plasma target 133 that is positioned near, but typically spaced apart from, the guide distal end 122D of the energy guide 122A, and the photoacoustic transducer 154 that is in optical communication with a surface of the energy guide 122A.

When utilized, the plasma target 133 receives energy emitted from the guide distal end 122D of the energy guide 122A to generate plasma in the catheter fluid 132 within the balloon interior 146, which, in turn, causes the creation of plasma bubbles (cavitation bubbles) and/or pressure waves that can be directed away from the surface of the energy guide 122A and toward the balloon wall 130. Additionally, or in the alternative, when utilized, the photoacoustic transducer 154 converts light energy into an acoustic wave that extends away from the surface of the energy guide 122A.

In certain embodiments, the diverting structures that can be incorporated into the energy guides 122A, can additionally and/or alternatively be incorporated into the design of the emitter assembly 129 and/or the plasma target 133 for purposes of directing and/or concentrating acoustic and mechanical energy toward specific areas of the balloon wall 130 in contact with the vascular lesions 106A at the treatment site 106 to impart pressure onto and induce fractures in such vascular lesions 106A. It is appreciated that the emitters 131 and/or the plasma target 133 can be designed, positioned and oriented in any suitable manner to direct the acoustic and/or mechanical energy toward any desired portion of the balloon wall 130 that is substantially adjacent to the vascular lesions 106A, such as a CTO, at the treatment site 106.

As so described, with each of the energy guides 122A potentially including one or more photoacoustic transducers 154 and/or one or more diverters at or near the guide distal end 122D and within the balloon interior 146, it is appreciated that each energy guide 122A can be utilized to help define one or more emitters 131 for purposes of directing energy toward any desired portion of the balloon wall 130 that is substantially adjacent to the treatment site 106.

The source manifold 136 can be positioned at or near the proximal portion 114 of the catheter system 100. In certain embodiments, the source manifold 136 can include one or more proximal end openings that can receive the one or more energy guides 122A of the energy guide bundle 122, the guidewire 112, an inflation conduit 140 that is coupled in fluid communication with the fluid pump 138, and/or a fluid outflow conduit 141 that is incorporated as part of the flushing system 135. Alternatively, one or more of the one or more energy guides 122A, the guidewire 112, the inflation conduit 140, and the fluid outflow conduit 141 can be provided as part of the catheter system 100 without specifically being directed through the source manifold 136.

The catheter system 100 can also include the fluid pump 138 that is configured to inflate the balloon 104 with the catheter fluid 132 as needed. As shown, the catheter system 100 can further include a valve 142 that can be utilized as part of the flushing system 135 and that is configured to control and/or regulate the outflow of catheter fluid 132 from within the balloon 104 and through the fluid outflow conduit 141 during use of the flushing system 135.

As noted above, in the embodiment illustrated in FIG. 1, the system console 123 includes one or more of the energy source 124, the power source 125, the system controller 126, and the GUI 127. Alternatively, the system console 123 can include more components or fewer components than those specifically illustrated in FIG. 1. For example, in certain non-exclusive alternative embodiments, the system console 123 can be designed without the GUI 127. Still alternatively, one or more of the energy source 124, the power source 125, the system controller 126, and the GUI 127 can be provided within the catheter system 100 without the specific need for the system console 123.

As shown, the system console 123, and the components included therewith, is operatively coupled to the catheter 102, the energy guide bundle 122, and the remainder of the catheter system 100. For example, in some embodiments, as illustrated in FIG. 1, the system console 123 can include a console connection aperture 148 (also sometimes referred to generally as a “socket”) by which the energy guide bundle 122 is mechanically coupled to the system console 123. In such embodiments, the energy guide bundle 122 can include a guide coupling housing 150 (which can generally include one or more ferrules) that houses a portion, such as at least the guide proximal end 122P, of each of the energy guides 122A. At least a portion of the guide coupling housing 150 is configured to fit and be selectively retained within the console connection aperture 148 to provide the mechanical coupling between the energy guide bundle 122 and the system console 123, as well as helping to provide an optical coupling between the energy source 124 and the energy guides 122A of the energy guide bundle 122.

The energy guide bundle 122 can also include a guide bundler 152 (or “shell”) that brings each of the individual energy guides 122A closer together so that the energy guides 122A and/or the energy guide bundle 122 can be in a more compact form as it extends as part of the catheter 102 into the blood vessel 108 during use of the catheter system 100.

The energy source 124 can be selectively and/or alternatively coupled in optical communication with each of the energy guides 122A, such as to the guide proximal end 122P of each of the energy guides 122A, in the energy guide bundle 122. In particular, the energy source 124 is configured to generate energy in the form of a source beam 124A, such as a pulsed source beam, that can be selectively and/or alternatively directed to and received by each of the energy guides 122A in the energy guide bundle 122 as an individual guide beam 124B. Alternatively, the catheter system 100 can include more than one energy source 124. For example, in one non-exclusive alternative embodiment, the catheter system 100 can include a separate energy source 124 for each of the energy guides 122A in the energy guide bundle 122.

The energy source 124 can have any suitable design. In certain embodiments, the energy source 124 can be configured to provide sub-millisecond pulses of energy from the energy source 124 that are focused onto a small spot in order to couple it into the guide proximal end 122P of the energy guide 122A. Such pulses of energy are then directed and/or guided along the energy guides 122A to a location within the balloon interior 146 of the balloon 104, thereby inducing plasma formation in the catheter fluid 132 within the balloon interior 146 of the balloon 104.

In many embodiments, plasma generation can occur via the plasma target 133, which can include and/or incorporate any suitable structure and can be positioned and oriented in any suitable manner at or near the guide distal end 122D of the energy guide 122A. In particular, the energy emitted from at or near the guide distal end 122D of the energy guide 122A is directed toward and contacts and energizes material of the plasma target 133 for purposes of generating plasma in the catheter fluid 132 within the balloon interior 146. The plasma generation ionizes and superheats the surrounding catheter fluid 132 and thus causes rapid inertial bubble formation, and imparts pressure waves upon the treatment site 106 to provide the fracture force onto the vascular lesions 106A at the treatment site 106. An exemplary plasma-induced bubble 134 is illustrated in FIG. 1.

More specifically, the generation of the plasma bubble 134 creates an outwardly emanating pressure wave throughout the catheter fluid 132 that impacts the balloon wall 130. The impact to the balloon wall 130 causes the balloon 104 to forcefully disrupt and/or fracture the vascular lesions 106A at the treatment site 106. Stated in another manner, the associated rapid formation of the plasma bubble 134 and resulting localized catheter fluid 132 velocity within the balloon 104 transfers mechanical energy though the incompressible catheter fluid 132 to impart a fracture force on the vascular lesions 106A at the treatment site 106. The rapid change in momentum of the catheter fluid 132 upon hitting the balloon wall 130 is known as hydraulic shock, or water hammer. The change in momentum of the catheter fluid 132 is transferred as a fracture force to the vascular lesion 106A which is opposed to the balloon wall 130.

In many embodiments, the present invention utilizes a laser light source or other suitable light source as the energy source 124, and is configured to shine laser light energy onto the plasma target 133 to cause plasma generation via interaction with the plasma target material rather than optical breakdown of the catheter fluid 132. This moves the plasma creation away from the guide distal end 122D of the energy guide122A (which can be an optical fiber in some embodiments). This can be accomplished by positioning the plasma target 133 away from the guide distal end 122D of the energy guide 122A to absorb the light energy and convert it into a plasma at some distance away from the guide distal end 122D of the energy guide 122A. By positioning the plasma target 133 away from the guide distal end 122D of the energy guide 122A, damage to the energy guide 122A from the plasma bubble 134 is less likely to occur than if the plasma bubble 134 was generated at or more proximate the guide distal end 122D of the energy guide 122A. Stated another way, the presence of the plasma target 133, and positioning the plasma target 133 away from the guide distal end 122D of the energy guide 122A, causes the plasma bubble 134 to in turn be generated away from the guide distal end 122D of the energy guide 122A, reducing the likelihood of damage to the energy guide 122A.

The plasma target 133 can be formed from any suitable materials that are configured to generate the desired plasma in the catheter fluid 132 within the balloon interior 146 when the energy is directed from the guide distal end 122D of the energy guide 122A to contact the plasma target 133. For example, in certain non-exclusive embodiments, the plasma target 133 can be formed, at least in part, from one or more metallics and/or metal alloys having relatively high melting temperatures, such as titanium, stainless steel, tungsten, tantalum, platinum, molybdenum, niobium, iridium, etc. Alternatively, the plasma target 133 can be formed from at least one of magnesium oxide, beryllium oxide, tungsten carbide, titanium nitride, titanium carbonitride, and titanium carbide. Still alternatively, the plasma target 133 can be formed from at least one of chemical vapor deposition (CVD) diamond and diamond. In other embodiments, the plasma target 133 can be formed from a transition metal, an alloy metal, or a ceramic material. Yet alternatively, in some embodiments, the plasma target 133 can be formed at least partially from a polymer, a polymeric material, and/or a plastic such as polyimide and nylon. Still alternatively, the plasma target 133 can be formed from any other suitable materials.

The plasma target 133 can have any suitable design, and can be positioned and oriented in any suitable manner such that the generated plasma, and the subsequently generated acoustic waves and/or pressure waves are directed as desired toward the vascular lesions 106A at the treatment site 106. For example, in certain embodiments, the plasma target 133 can be designed, positioned and oriented such that the emitter 131 functions as a forward-firing emitter, noted by arrow 145A, so as to better enable treatment of a CTO that may be encompassed and/or included within the treatment site 106. Alternatively, in other embodiments, the plasma target 133 can be designed, positioned and oriented in another suitable manner, such that the emitter 131 functions as other than a forward-firing emitter (a lateral-firing emitting, or emitter that fires at another suitable angle relative to the longitudinal axis 144 of the catheter shaft 110).

In various non-exclusive alternative embodiments, the sub-millisecond pulses of energy from the energy source 124 can be delivered to the treatment site 106 at a pulse frequency of between approximately one hertz (Hz) and 5000 Hz, between approximately 30 Hz and 1000 Hz, between approximately ten Hz and 100 Hz, or between approximately one Hz and 30 Hz. Alternatively, the sub-millisecond pulses of energy can be delivered to the treatment site 106 at a pulse frequency that can be greater than 5000 Hz or less than one Hz, or any other suitable range of pulse frequencies.

It is appreciated that although the energy source 124 is typically utilized to provide pulses of energy, the energy source 124 can still be described as providing a single source beam 124A, such as a single pulsed source beam.

The energy sources 124 suitable for use can include various types of light sources including lasers and lamps. For example, in certain non-exclusive embodiments, the energy source 124 can be an infrared laser that emits energy in the form of pulses of infrared light. Alternatively, as noted above, the energy sources 124 can include any suitable type of energy source.

Suitable lasers can include short pulse lasers on the sub-millisecond timescale. In some embodiments, the energy source 124 can include lasers on the nanosecond (ns) timescale. The lasers can also include short pulse lasers on the picosecond (ps), femtosecond (fs), and microsecond (µs) timescales. It is appreciated that there are many combinations of laser wavelengths, pulse widths and energy levels that can be employed to achieve plasma and the associated cavitation bubbles in the catheter fluid 132 of the catheter 102. In various non-exclusive alternative embodiments, the pulse widths can include those falling within a range including from at least ten ns to 3000 ns, at least 20 ns to 100 ns, or at least one ns to 500 ns. Alternatively, any other suitable pulse width range can be used.

Exemplary nanosecond lasers can include those within the UV to IR spectrum, spanning wavelengths of about ten nanometers (nm) to one millimeter (mm). In some embodiments, the energy sources 124 suitable for use in the catheter systems 100 can include those capable of producing light at wavelengths of from at least 750 nm to 2000 nm. In other embodiments, the energy sources 124 can include those capable of producing light at wavelengths of from at least 700 nm to 3000 nm. In still other embodiments, the energy sources 124 can include those capable of producing light at wavelengths of from at least 100 nm to ten micrometers (µm). Nanosecond lasers can include those having repetition rates of up to 200 kHz.

In some embodiments, the laser can include a Q-switched thulium:yttrium- aluminum-garnet (Tm:YAG) laser. In other embodiments, the laser can include a neodymium:yttrium-aluminum-garnet (Nd:YAG) laser, holmium:yttrium-aluminum-garnet (Ho:YAG) laser, erbium:yttrium-aluminum-garnet (Er:YAG) laser, excimer laser, helium- neon laser, carbon dioxide laser, as well as doped, pulsed, fiber lasers.

In still other embodiments, the energy source 124 can include a plurality of lasers that are grouped together in series. In yet other embodiments, the energy source 124 can include one or more low energy lasers that are fed into a high energy amplifier, such as a master oscillator power amplifier (MOPA). In still yet other embodiments, the energy source 124 can include a plurality of lasers that can be combined in parallel or in series to provide the energy needed to create the plasma bubble 134 in the catheter fluid 132.

The catheter system 100 can generate acoustic waves and/or pressure waves having maximum pressures in the range of at least one megapascal (MPa) to 100 MPa. The maximum pressure generated by a particular catheter system 100 will depend on the energy source 124 and the various parameters of the energy pulses from the energy source 124, the absorbing material, the bubble expansion, the propagation medium, the balloon material, and other factors. In various non-exclusive alternative embodiments, the catheter systems 100 can generate acoustic waves and/or pressure waves having maximum pressures in the range of at least approximately 0.1MPa to 50 MPa, at least approximately 0.1 MPa to 30 MPa, or at least approximately 15 MPa to 25 MPa.

The acoustic waves and/or pressure waves can be imparted upon the treatment site 106 from a distance within a range from at least approximately 0.1 millimeters (mm) to greater than approximately 25 mm extending longitudinally and/or radially from the energy guides 122A when the catheter 102 is placed at the treatment site 106. In various non-exclusive alternative embodiments, the acoustic waves and/or pressure waves can be imparted upon the treatment site 106 from a distance within a range from at least approximately ten mm to 20 mm, at least approximately one mm to ten mm, at least approximately 1.5 mm to four mm, or at least approximately 0.1 mm to ten mm extending longitudinally and/or radially from the energy guides 122A when the catheter 102 is placed at the treatment site 106. In other embodiments, the acoustic waves and/or pressure waves can be imparted upon the treatment site 106 from another suitable distance that is different than the foregoing ranges. In some embodiments, the acoustic waves and/or pressure waves can be imparted upon the treatment site 106 within a range of at least approximately 0.1 MPa to 30 MPa at a distance from at least approximately 0.1 mm to ten mm. In some embodiments, the acoustic waves and/or pressure waves can be imparted upon the treatment site 106 from a range of at least approximately 0.1 MPa to 25 MPa at a distance from at least approximately 0.1 mm to ten mm. Still alternatively, other suitable pressure ranges and distances can be used.

The power source 125 is electrically coupled to and is configured to provide necessary power to each of the energy source 124, the system controller 126, the GUI 127, the handle assembly 128, the fluid pump 138, and the flushing system 135. The power source 125 can have any suitable design for such purposes.

The system controller 126 is electrically coupled to and receives power from the power source 125. The system controller 126 is coupled to and is configured to control operation of each of the energy source 124, the GUI 127, the fluid pump 138, and the flushing system 135. The system controller 126 can include one or more processors or circuits for purposes of controlling the operation of at least the energy source 124, the GUI 127, the fluid pump 138, and the flushing system 135. For example, the system controller 126 can control the energy source 124 for generating pulses of energy as desired and/or at any desired firing rate or frequency of pulse delivery.

The system controller 126 can further be configured to control operation of other components of the catheter system 100 such as the positioning of the catheter 102 and/or the guide distal end 122D of the energy guides 122A adjacent to the treatment site 106, the inflation of the balloon 104 with the catheter fluid 132 via use of the fluid pump 138, the flowing of the catheter fluid 132 through the balloon 104 as part of the flushing system 135, etc. Further, or in the alternative, the catheter system 100 can include one or more additional controllers that can be positioned in any suitable manner for purposes of controlling the various operations of the catheter system 100. For example, in certain embodiments, an additional controller and/or a portion of the system controller 126 can be positioned and/or incorporated within the handle assembly 128.

The GUI 127 is accessible by the user or operator of the catheter system 100. The GUI 127 is electrically connected to the system controller 126. With such design, the GUI 127 can be used by the user or operator to ensure that the catheter system 100 is effectively utilized to impart pressure onto and induce fractures into the vascular lesions 106A at the treatment site 106. The GUI 127 can provide the user or operator with information that can be used before, during and after use of the catheter system 100. In one embodiment, the GUI 127 can provide static visual data and/or information to the user or operator. In addition, or in the alternative, the GUI 127 can provide dynamic visual data and/or information to the user or operator, such as video data or any other data that changes over time during use of the catheter system 100. In various embodiments, the GUI 127 can include one or more colors, different sizes, varying brightness, etc., that may act as alerts to the user or operator. Additionally, or in the alternative, the GUI 127 can provide audio data or information to the user or operator. The specifics of the GUI 127 can vary depending upon the design requirements of the catheter system 100, or the specific needs, specifications and/or desires of the user or operator.

As shown in FIG. 1, the handle assembly 128 can be positioned at or near the proximal portion 114 of the catheter system 100, and/or near the source manifold 136. In this embodiment, the handle assembly 128 is coupled to the balloon 104 and is positioned spaced apart from the balloon 104. Alternatively, the handle assembly 128 can be positioned at another suitable location.

The handle assembly 128 is attached to the catheter shaft 110 and is handled and used by the user or operator to operate, position and control the catheter 102. For example, in certain embodiments, the handle assembly 128 can be utilized to mechanically move or reposition the catheter shaft 110 such that the balloon 104 can be positioned substantially adjacent to the treatment site 106; and/or to mechanically move or reposition the guidewire lumen 118 relative to the catheter shaft 110 so as to selectively move the balloon distal end 104D of the balloon 104 between the first (inverted) configuration 105A and the second (extended) configuration 205B.

The design and specific features of the handle assembly 128 can vary to suit the design requirements of the catheter system 100. In the embodiment illustrated in FIG. 1, the handle assembly 128 is separate from, but in electrical and/or fluid communication with one or more of the system controller 126, the energy source 124, the fluid pump 138, the flushing system 135, and the GUI 127.

In certain embodiments, as noted above, the handle assembly 128 can include the configuration adjuster 155 positioned therewithin to enable the user of operator to selectively, mechanically move the guidewire lumen 118 relative to the catheter shaft 110 for purposes of moving the balloon 104 between the first (inverted) configuration 105A and the second (extended) configuration 205B. The configuration adjuster 155 can have any suitable design for purposes of selectively, mechanically moving the guidewire lumen 118 relative to the catheter shaft 110 so that the balloon 104 moves between the first (inverted) configuration 105A and the second (extended) configuration 205B.

In some embodiments, the handle assembly 128 can integrate and/or include at least a portion of the system controller 126 within an interior of the handle assembly 128. For example, as shown, in certain embodiments, the handle assembly 128 can include circuitry 156, which is electrically coupled between catheter electronics and the system console 123, and which can form at least a portion of the system controller 126. In some embodiments, the circuitry 156 can transmit such electrical signals or otherwise provide data to the system controller 126.

In one embodiment, the circuitry 156 can include a printed circuit board having one or more integrated circuits, or any other suitable circuitry. In an alternative embodiment, the circuitry 156 can be omitted, or can be included within the system controller 126, which in various embodiments can be positioned outside of the handle assembly 128, such as within the system console 123. It is understood that the handle assembly 128 can include fewer or additional components than those specifically illustrated and described herein.

In various embodiments, as noted above, the emitter assembly 129 includes and/or incorporates the at least one emitter station 143 and/or the at least one emitter 131 that is configured to transmit energy from the energy source 124 into the balloon interior 146 so that plasma and/or pressure waves are generated in the catheter fluid 132 within the balloon interior 146. Each emitter 131 includes the guide distal end 122D of one of the energy guides 122A and the corresponding plasma target 133 that is positioned near, but typically spaced apart from, the guide distal end 122D. As so defined, the emitters 131, and/or the components thereof, are configured to direct and/or concentrate energy generated in the catheter fluid 132 within the balloon interior 146 so as to impart pressure onto and induce fractures in vascular lesions 106A at the treatment site 106.

As noted above, in certain embodiments, the emitter 131 can function as a forward-firing emitter, which can be better suited for imparting the desired forces and/or energy to the chronic total occlusion (CTO) that is being treated at the treatment site 106. Alternatively, the emitter 131 can be configured to impart the desired forces and/or energy in any suitable direction toward the vascular lesions 106A at the treatment site 106. Stated in another manner, the emitter 131 can function as other than a forward-firing emitter, such as a lateral-firing emitter, or an emitter that imparts the desired forces and/or energy at any suitable angle relative to the longitudinal axis 144 of the catheter shaft 110.

The flushing system 135 is configured to selectively minimize contamination within the balloon 104. In particular, in certain embodiments, the flushing system 135 is used to flush used/contaminated catheter fluid 132 (inflation media) out of the balloon 104 by enabling the catheter fluid 132 to flow into the balloon 104 via a particular fluid inflow path (including the inflation conduit 140 and the inflation lumen 359 that are in fluid communication with one another so that the catheter fluid 132 is directed into the balloon interior 146), through the balloon 104, and then out of the balloon 104 via a particular fluid outflow path (the catheter fluid 132 flowing out of the balloon interior 146 and into and through at least a portion of the catheter shaft 110 before being directed into and/or through the fluid outflow conduit 141). In some embodiments, the outflow path for the flushing system 135 through the fluid outflow conduit 141 can be controlled and/or regulated via use of the valve 142, such as a one-way valve in certain embodiments, or other means for controlling the release of used catheter fluid 132. More particularly, as shown in FIG. 1, at the proximal portion 114 of the catheter system 100, the fluid outflow path can lead to the valve 142, which controls selective release of the used catheter fluid 132 out of the catheter system 100.

After treatment cycles using the emitters 131, the catheter fluid 132 (inflation media) is cycled into and back out of the balloon 104 to thereby minimize contamination within the balloon 104. Within the proximal portion 114 of the catheter system 100, the fluid outflow conduit 141 can lead to the valve 142 (or other means for controlling the release of used catheter fluid 132) out of the catheter system 100. It is appreciated that the valve 142 can have any suitable design for purposes of effectively controlling release of the used catheter fluid 132 out of the control system 100. For example, in some embodiments, the valve 142 can be a one-way valve that only allows the catheter fluid 132 to flow one-way therethrough, i.e. away from the balloon 104 and out of the catheter system 100. In one such embodiment, the valve 142 can be a check valve that opens automatically when the fluid pressure within the fluid outflow conduit 141 reaches a certain pre-established threshold. In another such embodiment, the valve 142 can be manually opened and closed to enable the selective release of the used catheter fluid 132. Alternatively, the valve 142 can have another suitable design that enables the selective release of used catheter fluid 132 from within the balloon 104 and the catheter system 100.

As with all embodiments illustrated and described herein, various structures may be omitted from the figures for clarity and ease of understanding. Additionally, the figures may include certain structures that can be omitted without deviating from the intent and scope of the invention. It is further recognized that the structures included in the various figures shown and described herein are not necessarily drawn to scale for ease of viewing and/or understanding.

FIG. 2A is another simplified schematic cross-sectional view illustration of the catheter system 100 illustrated in FIG. 1, which is again configured to treat the vascular lesions 106A at the treatment site 106 within or adjacent to the vessel wall 108A of the blood vessel 108, or within or adjacent to a heart valve, within the body 107 of the patient 109. In particular, FIG. 2A again illustrates that the catheter system 100 can include (i) the catheter 102 including one or more of the balloon 104, the catheter shaft 110, the guidewire 112, the guidewire lumen 118, the energy guide bundle 122 including the one or more energy guides 122A, the source manifold 136, the fluid pump 138, the handle assembly 128, the emitter assembly 129 that includes and/or incorporates the at least one emitter station 143 and/or the at least one emitter 131, and the flushing system 135; and (ii) the system console 123 including one or more of the energy source 124, the power source 125, the system controller 126, and the GUI 127.

As FIG. 2A is actually illustrating the same catheter system 100 as has been illustrated and described in detail in relation to FIG. 1, it is appreciated that the catheter 102, the balloon 104, the catheter shaft 110, the guidewire 112, the guidewire lumen 118, the energy guide bundle 122 including the one or more energy guides 122A, the source manifold 136, the fluid pump 138, the handle assembly 128, the emitter assembly 129 including the at least one emitter station 143 and/or the at least one emitter 131, the flushing system 135, the system console 123, the energy source 124, the power source 125, the system controller 126, and the GUI 127 have the same overall design, functionality and interrelationships as was described herein above. Thus, a detailed description of such components will not be repeated in relation to FIG. 2A.

As illustrated in FIG. 2A, the balloon 104 again is shown with the balloon wall 130 that defines the balloon interior 146 that is configured to receive and retain the catheter fluid 132 therein, the balloon proximal end 104P that is coupled and/or secured to the catheter shaft 110, and the balloon distal end 104D that is coupled and/or secured to the guidewire lumen 118.

However, in the particular implementation shown in FIG. 2A, the treatment site 106 no longer includes a CTO. Because the treatment site 106 no longer includes a CTO, the balloon 104 has been moved to the second (extended) configuration 205B, which can be better suited for treating the vascular lesions 106A at treatment sites 106 that do not include a CTO. More particularly, as noted above, it is appreciated that when the balloon 104 is in the second (extended) configuration 205B, the catheter system 100 can be better configured to treat vascular lesions 106A at the treatment site 106 that are positioned more radially within the blood vessel 108 and do not necessarily include a CTO.

As the balloon distal end 104D is coupled and/or secured to the guidewire lumen 118 and the balloon proximal end 104P is coupled and/or secured to the catheter shaft 110, selective, mechanical movement of the guidewire lumen 118 relative to the catheter shaft 110 causes the balloon 104 to selectively move between the first (inverted) configuration 105A (illustrated in FIG. 1) and the second (extended) configuration 205B. For example, selective, mechanical movement of the guidewire lumen 118 in a distal direction relative to the catheter shaft 110 can cause the balloon 104 to selectively move from the first (inverted) configuration 105A to the second (extended) configuration 205B.

As shown in FIG. 2A, with the balloon 104 now being in the second (extended) configuration 205B, the distal tip 104T of the balloon 104 is now configured as an extended tip, with the distal tip 104T extending more distally than the remainder of the balloon 104. Stated in another manner, the second (extended) configuration 205B is a configuration in which no portion of the balloon 104 is positioned more distally than where the balloon distal end 104D and/or the distal tip 104T is coupled and/or secured to the guidewire lumen 118.

As noted above, each emitter 131 includes the guide distal end 122D of one of the energy guides 122A and a corresponding plasma target 133 that is positioned near, but usually spaced apart from, the guide distal end 122D.

As further noted herein, a lateral-firing emitter is configured to fire more generally in a radial direction toward the balloon wall 130 of the balloon 104. In particular, when the emitters 131 are lateral-firing emitters, the plasma target 133 is configured, positioned and oriented such that the emitters 131 fire in a direction that is generally transverse, or at a suitable angle, relative to the longitudinal axis 144 of the catheter shaft 110. More specifically, as shown with regard to the second emitter station 143B, energy is emitted from the guide distal end 122D of the energy guide 122A and is directed toward the plasma target 133. The plasma target 133 thus receives the energy emitted from the guide distal end 122D of the energy guide 122A to generate plasma in the catheter fluid 132 within the balloon interior 146, which, in turn, causes the creation of plasma bubbles 134 and/or pressure waves that can be directed generally radially away from the surface of the energy guide 122A and toward the balloon wall 130. Thus, as illustrated, the plasma target 133 is designed, positioned and oriented such that the emitter 131 shown at the second emitter station 143B functions as a lateral-firing emitter, noted by arrow 245B, so as to better enable treatment of the vascular lesions 106A that are radially distributed about the blood vessel 108 at the treatment site 106.

In the implementation shown in FIG. 2A, because the treatment site 106 does not include a CTO, the first emitter station 143A with the forward-firing emitter 131 is not active and not firing, while the second emitter station 143B with the lateral-firing emitter 131 is active and firing.

In certain embodiments, as illustrated in FIG. 2A, when the balloon distal end 104D is in the second (extended) configuration 205B, the balloon 104 no longer includes the inverted region 347A (illustrated in FIG. 3A), but rather the same portion of the balloon 104 can be configured as an extended region 347B (illustrated in FIG. 3B) that extends distally from the flat portion 339 while still enabling the effective coupling and/or bonding of the balloon distal end 104D and/or the distal tip 104T to the guidewire lumen 118.

FIG. 2B is a simplified schematic cross-sectional view illustration of the catheter system 100 illustrated in FIG. 2A, with the balloon 104 again being shown in the second (extended) configuration 205B, but with the balloon 104 being in the deflated state. More particularly, as shown in FIG. 2B, in some embodiments, the balloon distal end 104D and/or the distal tip 104T can have a tapered design when the balloon 104 is in the second (extended) configuration 205B and the balloon 104 is in the deflated state. For example, when the balloon 104 is in the deflated state for purposes of positioning the catheter 102 adjacent to the vascular lesions 106A at the treatment site 106 within the body 107 of the patient 109, it can be preferred to have the balloon 104 in the second (extended) configuration 205B to enhance the crossing profile as the balloon 104 engages the vascular lesions 106A at the treatment site 106, and to provide a tapered distal tip 104T for better crossing. Then, once the balloon 104 is through the vascular lesions 106A and/or is positioned as desired at the treatment site 106, the balloon 104 can be selectively inflated with the catheter fluid 132 (illustrated in FIG. 2A) to move the balloon 104 from the deflated state (as shown in FIG. 2B) to the inflated state (as shown in FIG. 2A). The balloon 104 can then further be used for performing the desired treatment of the vascular lesions 106A. It is appreciated that the desired treatment of the vascular lesions 106A can be performed with the balloon 104 either in the second (extended) configuration 205B or in the first (inverted) configuration 105A. It is further appreciated that if it is desired to use the balloon 104 in the first (inverted) configuration 105A, such as in the treatment of vascular lesions 106A in the form of a CTO, it would be necessary to move the balloon 104 from the second (extended) configuration 205B to the first (inverted) configuration 105A in any suitable manner such as described in detail herein.

FIG. 3A is a simplified schematic view illustration of a portion of an embodiment of the catheter system 300, including an embodiment of the balloon 304 having features of the present invention, the balloon 304 being shown in the first (inverted) configuration 305A. FIG. 3A further illustrates a portion of an embodiment of a fluid flushing system 335 that can be incorporated within the catheter system 300.

The design of the catheter system 300 can be varied. In various embodiments, as illustrated in FIG. 3A, the catheter system 300 can include a catheter 302 including at least the balloon 304 having a balloon wall 330 that defines a balloon interior 346, a balloon proximal end 304P, and a balloon distal end 304D; a catheter shaft 310; a guidewire lumen 318; one or more energy guides 322A; an emitter assembly 329 including at least one emitter station 343 and/or at least one emitter 331; and the flushing system 335. Alternatively, in other embodiments, the catheter system 300 can include more components or fewer components than what is specifically illustrated and described herein. For example, certain components that were illustrated in FIG. 1, such as the guidewire 112, the source manifold 136, the fluid pump 138, the handle assembly 128, the system console 123, the energy source 124, the power source 125, the system controller 126, and the GUI 127, are not specifically illustrated in FIG. 3A for purposes of clarity, but would likely be included in any embodiment of the catheter system 300.

In this embodiment, the catheter shaft 310, the guidewire lumen 318, the one or more energy guides 322A, and the emitter assembly 329 are substantially similar in design and function to what was illustrated and described herein above in relation to FIG. 1. Accordingly, the catheter shaft 310, the guidewire lumen 318, the energy guides 322A, and the emitter assembly 329 will not be described again in detail.

As shown in FIG. 3A, in many embodiments, the balloon 304 is again configured to receive and retain a catheter fluid 332 within the balloon interior 346 that is utilized to selectively move the balloon 304 between a deflated state and an inflated state, such as described herein above. As further shown in FIG. 3A, the balloon proximal end 304P is again coupled and/or secured to the catheter shaft 310, and the balloon distal end 304D is again coupled and/or secured to the guidewire lumen 318.

In this embodiment, with the balloon 304 being shown in the first (inverted) configuration 305A, the balloon 304 is generally bullnose-shaped having a rounded, forward perimeter 337 having a radius of curvature 337R, and a substantially flat portion 339 that extends radially inwardly from the forward perimeter 337. As noted above, the flat portion 339 of the balloon 304 at or near the balloon distal end 304D enables a larger surface area of the balloon 304 to be positioned substantially directly adjacent to the treatment site 106 (illustrated in FIG. 1), which, in many instances, can encompass and/or include a chronic total occlusion (or “CTO”).

In some embodiments, it is desired that the radius of curvature 337R for the forward perimeter 337 at or near the balloon distal end 304D be relatively small so as to enable a larger flat portion 339 that can be positioned substantially adjacent to the treatment site 106. In many embodiments, the radius of curvature 337R for the forward perimeter 337 can be less than approximately five millimeters, less than approximately four millimeters, less than approximately three millimeters, less than approximately two millimeters, less than approximately one millimeter, less than approximately 0.9 millimeters, less than approximately 0.8 millimeters, less than approximately 0.7 millimeters, less than approximately 0.6 millimeters, less than approximately 0.5 millimeters, less than approximately 0.4 millimeters, less than approximately 0.3 millimeters, less than approximately 0.2 millimeters, or less than approximately 0.1 millimeters. In several embodiments, in order that a certain radius of curvature 337R exists for the forward perimeter 337 to avoid any potential sharp edges, a minimum value for the radius of curvature 337R can be at least approximately 0.01 millimeters, 0.02 millimeters, 0.03 millimeters, 0.04 millimeters, or 0.05 millimeters. For example, in certain non-exclusive alternative embodiments, the radius of curvature 337R for the forward perimeter 337 can be between approximately 0.01 millimeters and five millimeters. In other embodiments, the radius of curvature 337R for the forward perimeter 337 can be between approximately 0.02 millimeters and four millimeters. In still other embodiments, the radius of curvature 337R for the forward perimeter 337 can be between approximately 0.03 millimeters and three millimeters. In yet other embodiments, the radius of curvature 337R for the forward perimeter 337 can be between approximately 0.05 millimeters and one millimeter. Alternatively, the radius of curvature 337R for the forward perimeter 337 can be greater than or less than the specific ranges noted herein.

In certain embodiments, the balloon 304 can be configured such that the flat portion 339 at or near the balloon distal end 304D is sufficiently large to provide a greater amount of balloon surface area that can be positioned substantially directly adjacent to the vascular lesions 106A (illustrated in FIG. 1), such as the CTO, at the treatment site 106. For example, in some embodiments, the flat portion 339 can extend between approximately 0.1 millimeters and five millimeters. In other embodiments, the flat portion 339 can extend between approximately 0.2 millimeters and four millimeters. In still other embodiments, the flat portion 339 can extend between approximately 0.3 millimeters and three millimeters. Alternatively, the flat portion 339 can extend greater than or less than the specific ranges noted herein.

In certain embodiments, the balloon 304 can further include an inverted region 347A, at or near the balloon distal end 304D, that extends proximally from the flat portion 339 such that the balloon distal end 304D can be effectively coupled and/or secured to the guidewire lumen 318. More particularly, because the catheter system 300 in general, and the balloon 304 specifically, are configured to combat incidences in which the treatment site 106 encompasses and/or includes a CTO, in some embodiments, the guidewire lumen 318 does not extend as far distally as the balloon distal end 304D. Stated in another manner, a distal end 318D of the guidewire lumen 318 does not extend as far distally as the balloon 304, such as at the forward perimeter 337 and the flat portion 339 of the balloon 304. Stated in still another manner, the distal end 318D of the guidewire lumen 318 is positioned proximally in comparison to the forward perimeter 337 and the flat portion 339 of the balloon 304 at or near the balloon distal end 304D. Thus, with such design, the balloon 304 incorporates the inverted region 347A that enables the balloon 304 to be coupled and/or secured to the guidewire lumen 318, near or at the distal end 318D of the guidewire lumen 318.

The balloon 304 can be coupled and/or secured to the guidewire lumen 318 in any suitable manner. In the embodiment shown in FIG. 3A, in addition to the inverted region 347A incorporated into the balloon 304 near the balloon distal end 304D, the balloon 304 further includes a fold back region 349, adjacent to the inverted region 347A, that extends distally from the inverted region 347A such that a balloon inner surface 304I of the balloon 304 can be coupled and/or secured to the guidewire lumen 318 with a securing bond 317. The securing bond 317 can have any suitable design for purposes of effectively securing the balloon inner surface 304I of the balloon 304 to the guidewire lumen 318.

As illustrated and described herein, it is appreciated that such design, which enables the balloon inner surface 304I of the balloon 304 to be coupled and/or secured to the guidewire lumen 318, further better enables the balloon 304 to be quickly and easily moved between the first (inverted) configuration 305A (as shown in FIG. 3A) and the second (extended) configuration 305B (as shown in FIG. 3B). More particularly, as described herein, selective, mechanical movement of the guidewire lumen 318 relative to the catheter shaft 310 causes the balloon 304 to selectively move between the first (inverted) configuration 305A and the second (extended) configuration 305B. For example, in one movement, selective, mechanical movement of the guidewire lumen 318 in a distal direction relative to the catheter shaft 310 can cause the balloon 304 to selectively move from the first (inverted) configuration 305A to the second (extended) configuration 305B. In another movement, selective, mechanical movement of the guidewire lumen 318 in a proximal direction relative to the catheter shaft 310 can cause the balloon 304 to selectively move from the second (extended) configuration 305B to the first (inverted) configuration 305A.

With the balloon inner surface 304I of the balloon 304 being coupled and/or secured to the guidewire lumen 318, the balloon distal end 304D and/or the distal tip 304T merely need to unfold or fold slightly during movement between the first (inverted) configuration 305A and the second (extended) configuration 305B, without providing a pulling force against the securing bond 317. Stated in another manner, as the distal tip 304T moves between being configured as an inverted tip and being configured as an extended tip, the fold back region 349 of the balloon 304 at the balloon distal end 304D remains fully secured to the guidewire lumen 318 without any forces tending to unroll the fold back region 349, which would tend to peel the guide distal end 304D against the adhesive force of the securing bond 317. It is appreciated that such unrolling or peeling forces may otherwise be present if a balloon outer surface 304S is secured to the guidewire lumen 318 instead of the balloon inner surface 304I.

FIG. 3A further illustrates that, in some embodiments, the balloon 304 can have a balloon diameter 357 that is substantially equal to a shaft diameter 310D of the catheter shaft 310. As referred to herein, the balloon diameter 357 being substantially equal to the shaft diameter 310D is intended to signify that the balloon diameter 357 is within approximately 1%, 2%, 3%, 4%, 5%, 6%, 7%, 8%, 9%, or 10% of the shaft diameter 310D.

As noted above, in certain embodiments, the balloon diameter 357 (in the inflated state) can range from less than 0.5 millimeters (mm) up to 25 mm. In some embodiments, the balloon diameter 357 can range from at least one mm up to 14 mm. In other embodiments, the balloon diameter 357 can range from at least one mm up to two mm. Alternatively, the balloon diameter 357 can be greater than or less than the ranges specifically noted herein.

It is further appreciated that the flat portion 339 can encompass at least a certain percentage of the full balloon diameter 357. For example, in some embodiments, the flat portion 339 can encompass at least approximately 10% of the full balloon diameter 357. In other embodiments, the flat portion 339 can encompass at least approximately 15%, 20%, 25%, 30%, 35%, 40%, 45%, 50%, 55%, 60%, 65%, 70%, or 75% of the full balloon diameter 357. Alternatively, the flat portion 339 can encompass at least a different percentage of the full balloon diameter 357.

Stated in another manner, in some embodiments, a ratio of the full balloon diameter 357 to a length of the flat portion 339 can be less than approximately 10:1. In other embodiments, the ratio of the full balloon diameter 357 to the length of the flat portion 339 can be less than approximately 9:1, 8:1, 7:1, 6:1, 5:1, 4:1, 3:1, 2:1, or 1.5:1. Alternatively, the ratio of the full balloon diameter 357 to the length of the flat portion 339 can have another suitable value.

In certain embodiments, the balloon 304 can be formed from non-resilient material such that the balloon diameter 357 does not change during movement between the deflated state and the inflated state. More particularly, in such embodiments, the balloon diameter 357 can be substantially equal to the shaft diameter 310D regardless of whether the balloon 304 is in the deflated state or the inflated state. Alternatively, the balloon 304 can have a balloon diameter 357 that is different than (greater than or less than) the shaft diameter 310D. Still alternatively, the balloon 304 can be formed from a semi-resilient material or a resilient material, such that the balloon diameter 357 may change during movement between the deflated state and the inflated state.

As with the previous embodiments, the catheter system 300 and/or the emitter assembly 329 can include the at least one emitter station 343 and/or the at least one emitter 331 that is configured to transmit energy from the energy source 124 (illustrated in FIG. 1) into the balloon interior 346 in order to generate plasma and/or pressure waves in the catheter fluid 332 within the balloon interior 346. Each of the emitters 331 again includes a guide distal end 322D of one of the energy guides 322A, which is positioned within the balloon interior 346, and a corresponding plasma target 333 that is positioned near, but typically spaced apart from, the guide distal end 322D. During use of the catheter system 300, energy from the energy source 124 is directed toward and received by the energy guide 322A, is guided through the energy guide 322A, and is then emitted from at or near the guide distal end 322D of the energy guide 322A. The energy emitted from at or near the guide distal end 322D is directed toward and contacts and energizes the corresponding plasma target 333 for purposes of generating the plasma in the catheter fluid 332 within the balloon interior 346. The plasma generation ionizes and superheats the surrounding catheter fluid 332 and thus causes rapid inertial bubble formation, and imparts pressure waves upon the treatment site 106 to provide the fracture force onto the vascular lesions 106A at the treatment site 106. Thus, with such design, the emitters 331, and/or the components thereof, are configured to direct and/or concentrate energy generated in the catheter fluid 332 within the balloon interior 346 so as to impart pressure onto and induce fractures in the vascular lesions 106A, such as in the form of a CTO, at the treatment site 106.

The plasma target 333 can have any suitable design, and can be positioned and oriented in any suitable manner such that the generated plasma, and the subsequently generated acoustic waves and/or pressure waves are directed as desired toward the vascular lesions 106A at the treatment site 106. As noted above, in certain embodiments, the plasma target 333 can be configured, positioned and/or oriented such that the emitter 331 operates as a forward-firing emitter, noted by arrow 345A, in which energy is directed substantially parallel to a longitudinal axis 344 of the catheter shaft 310 and/or toward the flat portion 339 at the balloon distal end 304D. As previously described, the forward-firing emitter can be more effective for treating a chronic total occlusion at the treatment site 106. Alternatively, the plasma target 333 can have another suitable structural design and/or can be positioned and oriented in another suitable manner, such that the emitter 331 can function other than as a forward-firing emitter (such as a generally lateral-firing emitter). Still alternatively, the catheter system 300 and/or the emitter assembly 329 can include a mix of both forward-firing emitters and lateral-firing emitters.

In some embodiments, such as shown in FIG. 3A, the catheter system 300 and/or the emitter assembly 329 can include a first emitter station 343A at a first longitudinal position within the balloon interior 346 that is nearer to the balloon distal end 304D, which can include at least one emitter 331 that is configured to operate as a forward-firing emitter. Additionally, in certain embodiments, the catheter system 300 and/or the emitter assembly 329 can further include a second emitter station 343B at a second longitudinal position within the balloon interior 346 that is farther from the balloon distal end 304D than the first emitter station 343A, which can include at least one emitter 331 that is configured to operate as a lateral-firing (non-forward-firing) emitter. It is appreciated that the functioning of the emitter 331 at the second emitter station 343B as a lateral-firing (non-forward-firing) emitter is illustrated more clearly in FIG. 3B.

As noted, in this embodiment, the balloon 304 is generally bullnose-shaped when in the first (inverted) configuration 305A, including the forward perimeter 337, at or near the balloon distal end 304D, having a particular radius of curvature 337R, as well as the generally flat portion 339 that extends radially inwardly from the forward perimeter 337. As referred to herein, the rounded, forward perimeter 337 and the flat portion 339 that extends radially inwardly therefrom can be said to define a balloon distal region 304R that encompasses much of the balloon 304 at or near the balloon distal end 304D. In many embodiments, the emitters 331 that are incorporated within the emitter assembly 329 are positioned proximally relative to the balloon distal region 304R. Alternatively, the emitters 331 can be positioned in a different manner relative to the balloon distal region 304R.

As noted above, the flushing system 335 is configured to selectively minimize contamination within the balloon 304. In particular, in certain embodiments, the flushing system 335 is used to flush used/contaminated catheter fluid 332 out of the balloon 304 by enabling the catheter fluid 332 to flow (i) into the balloon 304 via the inflation conduit 140 (illustrated in FIG. 1) and the inflation lumen 359 (a fluid inflow direction is illustrated generally with an arrow 361), (ii) through the balloon 304, and (iii) then out of the balloon 304 via the fluid outflow conduit 141 (illustrated in FIG. 1) (a fluid outflow direction is illustrated generally with an arrow 363). In some embodiments, as described above, the outflow path for the flushing system 335 can be controlled via use of the valve 142 (illustrated in FIG. 1), such as a one-way valve in certain embodiments, or other means for controlling the release of used catheter fluid 332. More particularly, the outflow path into and through at least a portion of the catheter shaft 110 and into the fluid outflow conduit 141 can lead to the valve 142, which controls selective release of the used catheter fluid 332 out of the catheter system 300.

FIG. 3B is a simplified schematic view illustration of the portion of the catheter system 300 illustrated in FIG. 3A, with the balloon 304 now being shown in the second (extended) configuration 305B. In particular, FIG. 3B illustrates the balloon 304 having been moved to the second (extended) configuration 305B, such that the distal tip 304T of the balloon 304 is now configured as an extended tip that extends distally away from the flat portion 339 so as to be positioned more distally than the remainder of the balloon 304. As noted above, when the balloon 304 is in the second (extended) configuration 305B, the catheter system 300 is generally better suited to treat vascular lesions 106A (illustrated in FIG. 1) at the treatment site 106 (illustrated in FIG. 1) that are radially distributed relative to the balloon 304 and do not include a CTO.

While FIG. 3B again illustrates that the catheter system 300 can include the catheter 302 including at least the balloon 304 having a balloon wall 330 that defines a balloon interior 346, a balloon proximal end 304P, and a balloon distal end 304D; the catheter shaft 310; the guidewire lumen 318; the one or more energy guides 322A; the emitter assembly 329 including the at least one emitter station 343 and/or the at least one emitter 331; and the flushing system 335, since this is actually the same catheter system 300 that was illustrated and described in relation to FIG. 3A, these components are the same as what has been illustrated and described in relation to FIG. 3A. Thus, a detailed description of such components will not be repeated in relation to FIG. 3B.

As shown in FIG. 3B, the balloon 304 is again configured to receive and retain the catheter fluid 332 within the balloon interior 346 that is utilized to selectively move the balloon 304 between the deflated state and the inflated state, such as described herein above. As further shown in FIG. 3B, the balloon proximal end 304P is again coupled and/or secured to the catheter shaft 310, and the balloon distal end 304D is again coupled and/or secured to the guidewire lumen 318.

However, since the balloon 304 is now in the second (extended) configuration 305B, the balloon 304 no longer includes the inverted region 347A (illustrated in FIG. 3A), but rather includes an extended region 347B in which at least a portion of the balloon distal end 304D and/or the distal tip 304T extends distally from the remainder of the balloon 304. As shown, with the extended region 349 extending distally from the flat portion 339, the balloon distal end 304D and/or the distal tip 304T is still effectively coupled and/or secured to the guidewire lumen 318. More particularly, the previous inverted region 347A and fold back region 349 (illustrated in FIG. 3A) have now been extended distally so as to comprise the extended region 347B, with the balloon inner surface 304I still being coupled and/or secured to the guidewire lumen 318 via the securing bond 317.

With the balloon 304 now in the second (extended) configuration 305B, the catheter system 300 and/or the emitter assembly 329 can again include the at least one emitter station 343 and/or the at least one emitter 331 that is configured to transmit energy from the energy source 124 (illustrated in FIG. 1) into the balloon interior 346 in order to generate plasma and/or pressure waves in the catheter fluid 332 within the balloon interior 346. Moreover, as shown, the emitter 331 at the first emitter station 343A is not firing, and the emitter 331 at the second emitter station 343B is firing, with the emitter 331 at the second emitter station 343B functioning as a lateral-firing emitter, noted by arrow 345B.

It should be noted that, as used in this specification and the appended claims, the singular forms "a," "an," and "the" include plural referents unless the content and/or context clearly dictates otherwise. It should also be noted that the term “or” is generally employed in its sense including “and/or” unless the content or context clearly dictates otherwise.

It should also be noted that, as used in this specification and the appended claims, the phrase “configured” describes a system, apparatus, or other structure that is constructed or configured to perform a particular task or adopt a particular configuration. The phrase "configured" can be used interchangeably with other similar phrases such as arranged and configured, constructed and arranged, constructed, manufactured and arranged, and the like.

The headings used herein are provided for consistency with suggestions under 37 CFR 1.77 or otherwise to provide organizational cues. These headings shall not be viewed to limit or characterize the invention(s) set out in any claims that may issue from this disclosure. As an example, a description of a technology in the “Background” is not an admission that technology is prior art to any invention(s) in this disclosure. Neither is the “Summary” or “Abstract” to be considered as a characterization of the invention(s) set forth in issued claims.

The embodiments described herein are not intended to be exhaustive or to limit the invention to the precise forms disclosed in the detailed description provided herein. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

It is understood that although a number of different embodiments of the catheter system, the catheter, and/or the balloon have been illustrated and described herein, one or more features of any one embodiment can be combined with one or more features of one or more of the other embodiments, provided that such combination satisfies the intent of the present invention.

While a number of exemplary aspects and embodiments of the catheter system, the catheter, and/or the balloon have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope, and no limitations are intended to the details of construction or design herein shown.

What is claimed is:

1. A catheter comprising:
a catheter shaft;
a guidewire lumen that is positioned at least partially within the catheter shaft, the guidewire lumen being substantially rigid and having a distal end;
a balloon including a balloon proximal end that is coupled to the catheter shaft, a balloon distal end that is secured to the guidewire lumen, and a balloon wall that defines a balloon interior, the balloon being configured to retain a catheter fluid within the balloon interior, the balloon being selectively expandable with the catheter fluid to move to an expanded state wherein the balloon wall is positionable substantially adjacent to a treatment site, the balloon being selectively movable between an inverted configuration in which at least a portion of the balloon extends farther distally than the distal end of the guidewire lumen, and an extended configuration in which no portion of the balloon is positioned more distally than where the balloon distal end is secured to the guidewire lumen; and
a first energy guide that is configured to receive energy from an energy source, the first energy guide guiding the energy from the energy source into the balloon interior.

2. The catheter of claim 1, wherein mechanical movement of the guidewire lumen relative to the catheter shaft moves the balloon between the inverted configuration and the extended configuration.

3. The catheter of claim 1, wherein mechanical movement of the guidewire lumen relative to the catheter shaft in a distal direction moves the balloon from the inverted configuration to the extended configuration.

4. The catheter of claim 1, wherein mechanical movement of the guidewire lumen relative to the catheter shaft in a proximal direction moves the balloon from the extended configuration to the inverted configuration.

5. The catheter of claim 1, wherein when the balloon is in the inverted configuration, the balloon distal end is bullnose-shaped.

6. The catheter of claim 1, wherein when the balloon is in the inverted configuration, the balloon further includes an inverted region that extends proximally from a flat portion.

7. The catheter of claim 6, wherein when the balloon is in the inverted configuration, the balloon further includes a fold back region that extends distally from the inverted region.

8. The catheter of claim 7, wherein the balloon has a balloon inner surface and the fold back region is configured such that the balloon inner surface is secured to the guidewire lumen.

9. The catheter of claim 1, wherein when the balloon is in the extended configuration, the balloon further includes an extended region that extends distally from a flat portion.

10. The catheter of claim 1, wherein the catheter shaft has a shaft diameter, and the balloon has a balloon diameter that is approximately equal to the shaft diameter.

11. The catheter of claim 1, wherein the balloon is formed from a non-resilient material such that a balloon diameter remains constant when the balloon moves to the expanded state.

12. The catheter of claim 1, wherein the balloon has no tapered portions along a balloon length of the balloon from the balloon proximal end to the balloon distal end.

13. The catheter of claim 1, further comprising
a first emitter configured to emit the energy from the first energy guide so that a wave is generated in the catheter fluid within the balloon interior,
wherein the first emitter is a forward-firing emitter that directs energy from the wave toward the balloon distal end.

14. A catheter system comprising:
an energy source that generates energy;
a catheter shaft;
a guidewire lumen that is positioned at least partially within the catheter shaft, the guidewire lumen being substantially rigid and having a distal end;
a balloon including a balloon proximal end that is coupled to the catheter shaft, a balloon distal end that is secured to the guidewire lumen, and a balloon wall that defines a balloon interior, the balloon being configured to retain a catheter fluid within the balloon interior, the balloon being selectively expandable with the catheter fluid to move to an expanded state wherein the balloon wall is positionable substantially adjacent to a treatment site, the balloon being selectively movable between an inverted configuration in which at least a portion of the balloon extends farther distally than the distal end of the guidewire lumen, and an extended configuration in which no portion of the balloon is positioned more distally than where the balloon distal end is secured to the guidewire lumen; and
a first energy guide that is configured to receive the energy from the energy source, the first energy guide guiding the energy from the energy source into the balloon interior.

15. The catheter system of claim 14, wherein mechanical movement of the guidewire lumen relative to the catheter shaft moves the balloon between the inverted configuration and the extended configuration.

16. The catheter system of claim 14, wherein when the balloon is in the inverted configuration, the balloon distal end is bullnose-shaped.

17. The catheter system of claim 14, wherein when the balloon is in the inverted configuration, the balloon further includes an inverted region that extends proximally from a flat portion.

18. The catheter system of claim 14, wherein the catheter shaft has a shaft diameter, and the balloon has a balloon diameter that is approximately equal to the shaft diameter.

19. The catheter system of claim 14, further comprising
a first emitter configured to emit the energy from the first energy guide so that a wave is generated in the catheter fluid within the balloon interior,
wherein the first emitter is a forward-firing emitter that directs energy from the wave toward the balloon distal end.

20. A method for treating a treatment site within or adjacent to a vessel wall of a blood vessel, or within or adjacent to a heart valve, within a body of a patient, the method may comprise:
positioning a balloon of a catheter substantially adjacent to the treatment site;
moving the balloon between an inverted configuration in which at least a portion of the balloon extends farther distally than a distal end of a guidewire lumen of the catheter and an extended configuration in which no portion of the balloon is positioned more distally than where a balloon distal end is secured to the guidewire lumen; and
providing energy from an energy source to an energy guide with a distal end positioned in an interior of the balloon to create a wave within the balloon.
